(19) United States
(12) Patent Application Publication
(10) Pub. No.: US 2009/0011004 A1
(43) Pub. Date: Jan. 8, 2009

(54) IMPROVED CARRIERS FOR DELIVERY OF NUCLEIC ACID AGENTS TO CELLS AND TISSUES

(75) Inventors: Gordon John Lutz, Kennett Square, PA (US); Margaret Alison Wheatley, Media, PA (US)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignee: Philadelphia Health & Education Corp., D/B/A/ Drexel University of College of Medicine, Philadelphia, PA (US)

(21) Appl. No.: 12/159,286

(22) PCT Filed: Jan. 2, 2007
(86) PCT No.: PCT/US07/60016

§ 371 Date: Jun. 26, 2008

Related U.S. Application Data
(60) Provisional application No. 60/755,113, filed on Dec. 30, 2005.

(51) Int. Cl. A61K 47/48; A61K 31/7088; C12N 5/06; A61K 9/127; C08G 73/02

(52) U.S. Cl. 424/450; 525/54.2; 525/54.1; 424/78.27; 435/375; 514/44

(57) ABSTRACT

This invention relates to drug delivery and specifically to the preparation and use of functionalized carriers such as nanopolymers and nanovesicles for improved delivery of nucleic acid agents (NAA) to tissues and cells. These compounds have broad applicability for treating numerous diseases and disorders, including neurodegenerative and neuromuscular disorders. The concept encompasses preferably polymeric carriers for delivery of a class of oligonucleotides that modulate RNA splicing.

This application claims benefit of U.S. Provisional 60/755,113, filed Dec. 30, 2005, incorporated by reference herein in its entirety.

### BACKGROUND OF THE INVENTION

1. Field of Invention

This invention relates to drug delivery and specifically to the preparation and use of functionalized carriers such as nanopolymers and nanovesicles for improved delivery of nucleic acid agents to tissues and cells. These compounds have broad applicability for treating numerous diseases and disorders, including neurodegenerative and neuromuscular disorders.

2. Description of Related Art

Small oligonucleotides such as antisense oligoribonucleotides (AOs) and short interference RNA (siRNA) have seen a remarkable recent surge in popularity for basic and applied research. Over the past decade, a host of chemical modifications to the basic structure of AOs has greatly expanded their specificity, functionality, and resistance to degradation and thus markedly improved their potential usefulness. In particular, chemically-modified AOs are now seen as valuable agents for targeted down regulation of transcript expression, modulation of alternative splicing, and exon skipping (Bremmer-Bout, M et. al. (2004) Mol. Ther. V10. 232-240.; Dias, N et. al. (2002) Mol. Cancer Ther. V1.347-355.; Shi, F et. al. (2004) J. Control Release V97.189-209.; van Deutekom, J C et. al. (2003) Nat. Rev. Genet. V4. 774-783). Chemically modified AOs are also becoming increasingly valuable agents for gaining knowledge of protein function from genomic and proteomic studies (Goodchild, J (2004) Curr. Opin. Mol. Ther. V6. 120-128.; Ravichandran, L V et. al. (2004) Oligonucleotides. V14. 49-64.) as well as for treating diseases through modulation of pre-mRNA splicing, alternative splicing and exon skipping (Bremmer-Bout, M et. al. (2004) Mol. Ther. V10. 232-240.; Goodchild, J (2004) Curr. Opin. Mol. Ther. V6. 120-128.; Ravichandran, L V et. al. (2004) Oligonucleotides. V14.49-64.; Sazani, P et. al. (2003) J. Clin. Invest V112. 481-486.; van Deutekom, J C et. al. (2001) Hum. Mol. Genet. V10. 1547-1554).

Other studies have shown that AOs containing 2′-O-methyl modifications can facilitate exon skipping of dystrophin pre-mRNA and produce dystrophin expression in dystrophin-null mdx mice (artsma-Rus, A et. al. (2006) Mol. Ther. V14. 401-407.; Mann, C J et. al. (2002) J. Gene Med. V4. 644-654.) and in cultured cells of Duchenne muscular dystrophy (DMD) patients (Aartsma-Rus, A et. al. (2003) Hum. Mol. Genet. V12. 907-914.; van Deutekom, J C et. al. (2001) Hum. Mol. Genet. V10. 1547-1554). The AO-based approach for molecular therapy represents an attractive alternative to viral-based molecular therapies, as viral vectors continue to be plagued by complications arising from host immunogenic response, uncontrolled insertion into the host genome and spontaneous mutagenesis (Cerletti, M et. al. (2003) Gene Ther. V10. 750-757.; Gilchrist, S C et. al. (2002) Mol. Ther. V6. 359-368.; Jiang, Z et. al. (2004) Mol. Ther. V10. 688-696.; Marshall, E (1999) Science V286. 2244-2245.; Zaiss, A K et. al. (2005) Curr. Gene Ther. V5. 323-331). However, the lack of effective means to deliver small nucleic acid agents to target cells remains the foremost limitation to their usefulness as a non-viral alternative for molecular therapy.

A variety of small nucleic acid agents are applicable for use with polymer carriers as described herein, including 2′O-MeAOs, phosphorothioate oligonucleotides, siRNA, phosphodiester oligonucleotides, peptide nucleic acids, ribozymes, and other carrier-functionalized oligonucleotides.

Alone, small nucleic acid agents have very low transfection efficiency and are rapidly degraded by nucleases, necessitating the use of carrier molecules. Several reviews have defined the barriers that must be overcome for successful delivery of nucleic acid agents to target cell nuclei, and have outlined progress in carrier-mediated delivery to overcome those barriers (Dias, N et. al. (2002) Mol. Cancer Ther. V1. 347-355.; Shi, F et. al. (2004) J. Control Release V97. 189-209.; Wiethoff, C M et. al. (2003) J. Pharm. Sci. V92. 203-217). For carrier-mediated transfection of both AOs and siRNA, many studies have relied on cationic lipoplexes some of which are commercially available. Although significant progress has been made in improving lipoplexes for oligonucleotide delivery (Ulrich, A S (2002) Biosci. Rep. V22. 129-150), cytotoxicity and serum reactivity continue to hinder their usefulness, especially for in vivo applications.

Positively charged polymers have been used in non-viral delivery compositions for molecular therapy applications. Such polymers demonstrate a self-assembling property when mixed with nucleic acid agents due to ionic interactions. The cationic polymer polyethylenimine (PEI) is well-known as an efficient nucleic acid carrier due to formation of PEI-nucleotide complexes that show high transfection capacity and flexibility for addition of moieties that target specific entities on cell membranes and intracellular structures (Bieber, T et. al. (2002) J. Control Release V82.441-454.; Boussif, O et. al. (1995) Proc. Natl. Acad. Sci. U.S.A V92. 7297-7301.; Godbey, W T et. al. (1999) Proc. Natl. Acad. Sci. U.S.A V96. 5177-5181.; Kichler, A (2004) J. Gene Med. V6 Suppl 1. S3-10.; Petersen, H et. al. (2002) Bioconjug. Chem. V13. 845-854.; Petersen, H et. al. (2002) Macromolecules V35. 6867-6874.; Suh, J et. al. (2003) Proc. Natl. Acad. Sci. U.S.A V100. 3878-3882.; Thomas, M et. al. (2003) Appl. Microbiol. Biotechnol. V62. 27-34). PEI forms polyplexes with anionic nucleotides by charge coupling. In the absence of a targeting moiety cellular uptake of the polyplexes appears to occur by non-specific adsorptive endocytosis, although this process is not well understood. Escape from the endosomes is facilitated by protonation of amines on PEI, the so-called “proton sponge” effect (Akinc, A et. al. (2004) J. Gene Med. V; Boussif, O et. al. (1995) Proc. Natl. Acad. Sci. U.S.A V92. 7297-7301.; Hara-Chikuma, M et. al. (2004) Journal of Biological Chemistry V; Sonawane, N D et. al. (2003) Journal of Biological Chemistry V278. 44826-44831.; Thomas, M et. al. (2003) Appl. Microbiol. Biotechnol. V62.27-34). Once released from endosomes, nucleic acid agents may enter the nucleus by a process that likely requires dissociation from the polymer carrier.

The functionality of PEI as a nucleotide carrier is significantly improved by incorporating the nonionic linear polymer polyethylene glycol (PEG) into PEG-PEI copolymers (Kichler, A (2004) J. Gene Med. V6 Suppl 1. S3-10.; Petersen, H et. al. (2002) Macromolecules V35. 6867-6874.; Sung, S J et. al. (2003) Biol. Pharm. Bull. V26. 492-500). Complexation of PEG-PEI copolymers with small nucleic acid agents produces particles with a core-shell structure, whereby PEI and the electrostatically-coupled nucleic acid agent are oriented toward the particle core and the PEG chains form a brushlike corona, although the precise nature of this arrangement remains an open question. In general, PEG provides polyplexes with improved solubility, lower surface charge, diminished aggregation, lower cytotoxicity, and decreased opsonization with serum proteins in the bloodstream. However, these desirable properties may come at a cost of lower transfection efficiency due to both reduced membrane interaction and less efficient endosomal escape. The precise function of PEGylation may depend on many factors including PEG molecular weight, PEI molecular weight, stoichiometry of PEG grafting, type of nucleic acid agent, and environment.

Although PEG-PEI copolymers have been primarily used for plasmid delivery, other studies have provided strong evidence that PEGylated PEI can be adapted to function as an effective carrier for cellular delivery of small oligonucleotides (Brus, C et. al. (2004) Bioconjug. Chem. V15. 677-684.; Fischer, D et. al. (2004) Drug Metab Dispos. V32.983-992.; Jeong, J H et. al. (2003) J. Control Release V93. 183-191.; Kunath, K et. al. (2002) Pharm. Res. V19.810-817.; Schiffelers, R M et. al. (2004) Nucleic Acids Res. V32. e149-; Vinogradov, S et. al. (1999) Bioconjug. Chem. S V 10. 851-860.; Vinogradov, S V et. al. (1998) Bioconjug. Chem. V9. 805-812.; Vinogradov, S V et. al. (2004) Bioconjug. Chem. V15. 50-60). These and other studies have offered insights into the influence of the PEG chain length, extent of PEG grafting, and method of PEGylation on the polyplex structural properties, nuclease protection, complement activation, serum stability, transfection efficiency, and in vivo distribution.

Numerous functional groups that have been used in drug delivery may be applicable to use with cationic polymers to improve transfection capacity and delivery of small nucleic acids to tissues and cells. Some of these are described as follows.

Gold nanoparticles (GNPs), including colloidal gold, are versatile agents used for a wide variety of biomedical applications, including the delivery of genes and drugs (Hainfeld, J F et. al. (2000) J. Histochem. Cytochem. V48. 471-480). GNPs have previously been shown to improve cellular uptake and biocompatibility of polymeric nucleotide carriers (Hainfeld, J F et. al. (2000) J. Histochem. Cytochem. V48.471-480.; Thomas, M et. al. (2003) Proc. Natl. Acad. Sci. U.S.A V100. 9138-9143). Internalization of GNPs into various cell types including muscle cells has been demonstrated (Kaisto, T et. al. (1999) Exp. Cell Res. V253. 551-560.; Shukla, R et. al. (2005) Langmuir V21. 10644-10654.; Thomas, M et. al. (2003) Proc. Natl. Acad. Sci. U.S.A V100. 9138-9143). In addition to facilitating cellular uptake, GNPs are inherently non-cytotoxic and have been shown to reduce the production of reactive oxygen and nitrite species, and prevent stimulation of proinflammatory cytokines (Shukla, R et. al. (2005) Langmuir V21. 10644-10654). Previous studies have also shown that coating of polymers with gold particles significantly reduced platelet and monocyte activation and reduced bacterial adhesion, generally improving the biocompatibility of the polymers (Hsu, S H et. al. (2006) J. Biomed. Mater. Res. A V).

The protein transduction domain (PTD) of HIV-TAT (trans activator of transcription) consists of an arginine-rich 11 amino acid peptide, and has been exploited as a carrier for the in vivo delivery of a variety of compounds. Since the discovery that only 11 amino acids of TAT are required for transduction, the use of TAT PTD has increased significantly (Ho, A et. al. (2001) Cancer Res. V61.474-477.; Nagahara, H et. al. (1998) Nat. Med. V4. 1449-1452.; Toro, A et. al. (2006) J. Clin. Invest V116. 2717-2726). It is well established that fusion complexes of TAT-PTD-cargo are able to circulate in the bloodstream, escape the microvasculature and undergo cellular uptake in a variety of tissues including skeletal muscles and the myocardium (Brooks, H et. al. (2005) Adv. Drug Deliv. Rev. V57.559-577.; Moulton, H M et. al. (2003) Antisense Nucleic Acid Drug Dev. V13. 31-43). Nuclear localization sequences within TAT-PTD can also promote nuclear uptake of the fusion complexes (Toro, A et. al. (2006) J. Clin. Invest V116. 2717-2726).

Apolipoprotein E (ApoE) is a 34 kD low-density lipoprotein (LDL) binding protein. ApoE is composed of two structural domains, an LDL receptor (LDLR) binding domain, and a lipid binding carboxy terminal domain. It was recently shown that ApoE facilitates the delivery of drugs by utilizing both endocytotic and transcytotic mechanisms of transport (Kreuter, J et. al. (2002) J. Drug Target V10.317-325.; Kreuter, J (2004) J. Nanosci. Nanotechnol. V4.484-488.; Michaelis, K et. al. (2006) J. Pharmacol. Exp. Ther. V317. 1246-1253).

Albumin conjugation to ligands and nanoparticles has been demonstrated to improve the circulation half-life of drugs injected into the bloodstream (Dennis, M S et. al. (2002) Journal of Biological Chemistry V277. 35035-35043.; Robinson, D M et. al. (2006) Drugs V66. 941-948.) and has been used in drug delivery applications to facilitate transcytosis across the capillary endothelium to target cells (Gradishar, W J (2006) Expert. Opin. Pharmacother. V7. 1041-1053.; Hillaireau, H et. al. (2006) J. Nanosci. Nanotechnol. V6. 2608-2617). Albumin transcytosis and cellular uptake appear to be mediated by caveolin-dependent processes (Cohen, A W et. al. (2004) Physiol Rev. V84.1341-1379.; Vogel, S M et. al. (2001) Am. J. Physiol Lung Cell Mol. Physiol V281. L1512-L1522).

Due to its cationic nature there is an inherent tradeoff in the PEG-PEI-NAA delivery system between transfection capacity and biodistribution. On one hand the residual positive surface charge on PEG-PEI-NAA polyplexes is important for stimulating cellular uptake. On the other hand the positive surface charge acts to decrease circulation time in the blood and limits diffusional distribution in the tissue interstitium due to electrostatic interactions with negatively charged elements in the bloodsteam and interstium. A useful alternative approach would be to encapsulate PEG-PEI-NAA polyplexes within inert and degradable synthetic polymer nanovesicles to facilitate improved biodistribution to the interstitium of target tissues. This “encapsulation approach” may enhance NAA delivery of by protecting the NAA from enzymatic digestion and by shielding polyplex surface charge.

Poly(lactic-co-glycolic acid) (PLGA) polymers are biodegradable and biocompatible compounds that have been approved by the Food and Drug Administration and utilized in a wide variety of drug delivery applications (Bala, I et. al. (2004) Crit. Rev. Ther. Drig Carrier Syst. V21. 387-422.; Panyam, J et. al. (2004) Curr. Drug Deliv. V1. 235-247). The versatility of PLGA vesicles stems from the fact the macromolecular properties can be controlled by varying the synthesis components and conditions, providing vesicles with a broad size range and variable release kinetics (Astete, C E et. al. (2006) J. Biomater. Sci. Polym. Ed V17. 247-289). Additionally, PLGA vesicles contain free carboxyl groups that are useful for attaching various moieties to the surface that can improve performance. Despite their proven utility in many drug delivery studies, PLGA vesicles have been used only sparingly for the delivery of polymer-NAA compounds, and this has been restricted to micron size vesicles (De Rosa, G et. al. (2002) J. Pharm. Sci. V91. 790-799.; De Rosa, G et. al. (2003) Int. J. Pharm. V254. 89-93.; DeRosa G. et. al. (2003) Biomacromolecules. V4.529-536.; Howard, K A et. al. (2004) Biochim. Biophys. Acta V1674. 149-157.; Moffatt, S et. al. (2006) Int. J. Pharm. V321. 143-154).

All references cited herein are incorporated herein by reference in their entireties.

### BRIEF SUMMARY OF THE INVENTION

This invention relates to drug delivery and specifically to the preparation and use of functionalized carriers such as nanopolymers and nanovesicles for improved delivery of nucleic acid agents (NAA) to tissues and cells. These compounds have broad applicability for treating numerous diseases and disorders, including neurodegenerative and neuromuscular disorders. The concept encompasses preferably polymeric carriers for delivery of a class of oligonucleotides that modulate RNA splicing. The carrier-NAA compounds are engineered with improved functionality including: improved transfection capacity, enhanced stability, cell/tissue specific targeting, and controlled release properties. Further embodiments of the invention set forth that the synthetic NAA carriers comprise PEG-PEI copolymers adapted for specific delivery modalities. Still other embodiments include functionalization of the PEG-PEI copolymers with gold nanoparticles, peptide transduction and viral tropism sequences, antibodies, and other cell targeting/transport ligands. Still further embodiments comprise NAAs that specifically modulate RNA splicing at the target gene to rescue protein expression defects in diseases including Spinal Muscular Atrophy. A further embodiment of the invention provides for functionalized nanovesicles and microbubbles, preferably of PLGA, used as carriers for delivery of carrier-NAA compounds.

The invention provides a PEG-PEI-Nucleic Acid Agent (NAA) polyplex comprising a PEG-PEI copolymer, optionally comprising one or more functionalization moieties, and a NAA, wherein the NAA is associated with the copolymer by electrostatic interactions, wherein the PEI is a branched structure with a Molecular Weight (MW) from about 2 to about 25 kDa.

The invention provides a PEG-PEI-Nucleic Acid Agent (NAA) polyplex comprising a PEG-PEI copolymer, optionally comprising one or more functionalization moieties, and a NAA, wherein the NAA is associated with the copolymer by electrostatic interactions, wherein the MW of PEG may range from about 500 to about 5000 Da.

The invention provides a PEG-PEI-Nucleic Acid Agent (NAA) polyplex comprising a PEG-PEI copolymer, optionally comprising one or more functionalization moieties, and a NAA, wherein the NAA is associated with the copolymer by electrostatic interactions, wherein the number of PEG chains grafted per molecule of PEI may range from about 1 to about 25.

The invention provides a PEG-PEI-Nucleic Acid Agent (NAA) polyplex comprising a PEG-PEI copolymer, optionally comprising one or more functionalization moieties, and a NAA, wherein the NAA is associated with the copolymer by electrostatic interactions, wherein the molar ratio of PEI amines (N) to NAA phosphates (P) (N:P ratio) may range from about 1 to about 15.

The invention provides a PEG-PEI-Nucleic Acid Agent (NAA) polyplex comprising a PEG-PEI copolymer, optionally comprising one or more functionalization moieties, and a NAA, wherein the NAA is associated with the copolymer by electrostatic interactions comprising one or more functionalization moieties, further wherein the one or more functionalization moieties attached to the PEG-PEI copolymer has an effect selected from the group consisting of improving polyplex stability, improving polyplex biodistribution, improving polyplex tissue delivery, improving polyplex and/or NAA cellular uptake, and combinations thereof.

The invention provides a PEG-PEI-Nucleic Acid Agent (NAA) polyplex comprising a PEG-PEI copolymer, optionally comprising one or more functionalization moieties, and a NAA, wherein the NAA is associated with the copolymer by electrostatic interactions, further wherein the functionalization moiety is a member selected from the group consisting of gold nanoparticles (GNP), TAT-PTD and derivatives thereof, ApoE, albumin, antibody, antibody fragment, magnetic nanoparticle, iron oxide, transferrin, AAV tropism fragment, and combinations thereof.

The invention provides a PEG-PEI-Nucleic Acid Agent (NAA) polyplex comprising a PEG-PEI copolymer, optionally comprising one or more functionalization moieties, and a NAA, wherein the NAA is associated with the copolymer by electrostatic interactions, further wherein the NAA is selected from the group consisting of antisense oligoribonucleotide (AO), oligodeoxynucleotide (ODN), U7-snRNA, siRNA, shRNA, PNA, ribozyme, aptamer, nucleoside 5′ triphosphates, and combinations thereof.

The invention provides a PEG-PEI-Nucleic Acid Agent (NAA) polyplex comprising a PEG-PEI copolymer, optionally comprising one or more functionalization moieties, and a NAA, wherein the NAA is associated with the copolymer by electrostatic interactions, further wherein the NAA is an antisense oligoribonucleotide (AO), wherein one or more of the bases is chemically modified, and further wherein the chemical modification is a member selected from the group consisting of 2′O-methyl, phosphorothioate, 2′MEO, phosphodiester, and combinations thereof.

The invention provides a PEG-PEI-Nucleic Acid Agent (NAA) polyplex comprising a PEG-PEI copolymer, optionally comprising one or more functionalization moieties, and a NAA, wherein the NAA is associated with the copolymer by electrostatic interactions, wherein the NAA comprises a carrier-functionalized oligonucleotide (CFO) which comprises an AO hybridized to a partially complimentary carrier strand by Watson-Crick base pairing.

The invention provides a PEG-PEI-Nucleic Acid Agent (NAA) polyplex comprising a PEG-PEI copolymer, optionally comprising one or more functionalization moieties, and a NAA, wherein the NAA is associated with the copolymer by electrostatic interactions, wherein the NAA is a CFO and the carrier strand contains a targeting group which has an effect selected from the group consisting of increasing delivery of the AO to tissues, delivery of the AO across the microvasculature, cellular uptake of the AO, nuclear localization of the AO, and combinations thereof.

The invention provides a PEG-PEI-Nucleic Acid Agent (NAA) polyplex comprising a PEG-PEI copolymer, optionally comprising one or more functionalization moieties, and a NAA, wherein the NAA is associated with the copolymer by electrostatic interactions, wherein the NAA is a CFO and the carrier strand contains a targeting group which is a member selected from the group of TAT-PTD (and derivatives thereof), AAV tropism factor, NLS peptide, cell targeting peptide, and combinations thereof.

The invention provides a PEG-PEI-Nucleic Acid Agent (NAA) polyplex comprising a PEG-PEI copolymer, optionally comprising one or more functionalization moieties, and a NAA, wherein the NAA is associated with the copolymer by electrostatic interactions, further wherein PEI has a MW of about 2 kDa; PEG has a MW of about 550 Da; PEG:PEI molar ratio is about 10; and the N:P ratio is about 1 to about 5.

The invention provides a PEG-PEI-Nucleic Acid Agent (NAA) polyplex comprising a PEG-PEI copolymer, optionally comprising one or more functionalization moieties, and a NAA, wherein the NAA is associated with the copolymer by electrostatic interactions, further wherein PEI has a MW of about 2 kDa; PEG has a MW of about 5 kDa; PEG:PEI molar ratio is about 10; and the N:P ratio is about 1 to about 5. The invention provides a PEG-PEI-Nucleic Acid Agent (NAA) polyplex comprising a PEG-PEI copolymer, optionally comprising one or more functionalization moieties, and a NAA, wherein the NAA is associated with the copolymer by electrostatic interactions, further wherein PEI has a MW of about 25 kDa; PEG has a MW of about 5 kDa; PEG:PEI molar ratio is about 10; and the N:P ratio is about 2 to about 5.

The invention provides a PEG-PEI-Nucleic Acid Agent (NAA) polyplex comprising a PEG-PEI copolymer, optionally comprising one or more functionalization moieties, and a NAA, wherein the NAA is associated with the copolymer by electrostatic interactions, further wherein the functionalization moiety is a member selected from the group consisting of ligands, receptors, monoclonal antibodies, polyclonal antibodies, small molecule ligands, aptamers, and combinations thereof.

The invention provides a PEG-PEI-Nucleic Acid Agent (NAA) polyplex comprising a PEG-PEI copolymer, optionally comprising one or more functionalization moieties, and a NAA, wherein the NAA is associated with the copolymer by electrostatic interactions, further wherein the functionalization moiety binds to a protein which is a member selected from the group consisting of tumor-markers, integrins, cell surface receptors, transmembrane proteins, ion channels, membrane transport protein, enzymes, antibodies, and chimeric proteins.

The invention provides a PEG-PEI-Nucleic Acid Agent (NAA) polyplex comprising a PEG-PEI copolymer, optionally comprising one or more functionalization moieties, and a NAA, wherein the NAA is associated with the copolymer by electrostatic interactions, further wherein the NAA contains a 2′O-methyl or morpholino AO of sequence 5′-AUUCACUUUCAUAAUGCUGG-3′ (SEQ ID NO: 1) for specific inclusion of human exon 7 of the SMN2 gene, useful for treating Spinal Muscular Atrophy. The invention provides a PEG-PEI-Nucleic Acid Agent (NAA) polyplex comprising a PEG-PEI copolymer, optionally comprising one or more functionalization moieties, and a NAA, wherein the NAA is associated with the copolymer by electrostatic interactions, further wherein the NAA contains a 2′O-methyl or morpholino AO of sequence 5′-UCAAGGAAGAUGGCAUUUCU-3′ (SEQ ID NO: 2) for specific skipping of human exon 51 of the dystrophin gene, and is useful for treating Duchenne Muscular Dystrophy.

The invention provides a synthetic polymer nanovesicle encapsulating either PEG-PEI-NAA polyplex or NAA alone, wherein the nanovesicle optionally comprises surface modifications and attached moieties for delivery of NAA to tissues and cells.

The invention provides a synthetic polymer nanovesicle encapsulating either PEG-PEI-NAA polyplex or NAA alone, wherein the nanovesicle optionally comprises surface modifications and attached moieties for delivery of NAA to tissues and cells, further wherein the synthetic polymer is a member selected from the group consisting of poly(lactic acid) (PLA), poly(glycolic acid) (PGA), and poly(lactic-co-glycolic acid) (PLGA).

The invention provides a synthetic polymer nanovesicle encapsulating either PEG-PEI-NAA polyplex or NAA alone, wherein the nanovesicle optionally comprises surface modifications and attached moieties for delivery of NAA to tissues and cells.

The invention provides a synthetic polymer nanovesicle encapsulating either PEG-PEI-NAA polyplex or NAA alone, wherein the nanovesicle optionally comprises surface modifications and attached moieties for delivery of NAA to tissues and cells, further wherein the NAA is a member selected from the group consisting of antisense oligoribonucleotide (AO), oligodeoxynucleotide (ODN), U7-snRNA, siRNA, shRNA, PNA, ribozyme, aptamer, nucleoside 5′ triphosphates, and combinations thereof.

The invention provides a synthetic polymer nanovesicle encapsulating either PEG-PEI-NAA polyplex or NAA alone, wherein the nanovesicle optionally comprises surface modifications and attached moieties for delivery of NAA to tissues and cells, wherein the NAA is an antisense oligoribonucleotide (AO), and wherein one or more of the bases is chemically modified, and further wherein the chemical modification is a member selected from the group consisting of 2′O-methyl, phosphorthioate, 2′MEO, phosphodiester, and combinations thereof.

The invention provides a synthetic polymer nanovesicle encapsulating either PEG-PEI-NAA polyplex or NAA alone, wherein the nanovesicle optionally comprises surface modifications and attached moieties for delivery of NAA to tissues and cells, wherein the NAA is a CFO.

The invention provides a synthetic polymer nanovesicle encapsulating either PEG-PEI-NAA polyplex or NAA alone, wherein the nanovesicle optionally comprises surface modifications and attached moieties for delivery of NAA to tissues and cells, further wherein the surface properties of the synthetic polymer nanovesicle is modified by attachment of a compound selected from the group consisting of PEG, GNP, ApoE, transferrin, albumin, and combinations thereof.

The invention provides a synthetic polymer nanovesicle encapsulating either PEG-PEI-NAA polyplex or NAA alone, wherein the nanovesicle optionally comprises surface modifications and attached moieties for delivery of NAA to tissues and cells, further wherein the synthetic polymer nanovesicle is modified by attachment of a compound selected from the group consisting of magnetic nanoparticles, iron oxide, and combinations thereof.

The invention provides a synthetic polymer nanovesicle encapsulating either PEG-PEI-NAA polyplex or NAA alone, wherein the nanovesicle optionally comprises surface modifications and attached moieties for delivery of NAA to tissues and cells, further wherein the surface properties of the synthetic polymer nanovesicles are modified by attachment of compounds selected from the group consisting of TAT-PTD and derivatives thereof, AAV tropism factors, antibodies, antibody fragments, and combinations thereof.

The invention provides a synthetic polymer nanovesicle encapsulating either PEG-PEI-NAA polyplex or NAA alone, wherein the nanovesicle optionally comprises surface modifications and attached moieties for delivery of NAA to tissues and cells, further wherein the synthetic polymer nanovesicle is modified by attachment of functionalized PEG-PEI copolymers.

The invention provides a synthetic polymer nanovesicle encapsulating either PEG-PEI-NAA polyplex or NAA alone, wherein the nanovesicle optionally comprises surface modifications and attached moieties for delivery of NAA to tissues and cells, further wherein the PEG-PEI copolymer comprises PEI with a MW of about 200 to about 2500 kDa, PEG with a MW of about 200 to about 5000 Da, and the PEG:PEI ratio is about 1:25.

The invention provides a synthetic polymer nanovesicle encapsulating either PEG-PEI-NAA polyplex or NAA alone, wherein the nanovesicle optionally comprises surface modifications and attached moieties for delivery of NAA to tissues and cells, further wherein the PEG-PEI copolymer comprises a functionalization moiety selected from GNP, TAT-PTD and derivatives thereof, AAV tropism factor, NLS peptide, cell targeting peptide, cell penetrating peptides, and combinations thereof.

The invention provides a synthetic polymer nanovesicle encapsulating either PEG-PEI-NAA polyplex or NAA alone, wherein the nanovesicle optionally comprises surface modifications and attached moieties for delivery of NAA to tissues and cells, further wherein the mean diameter of the synthetic polymer nanovesicle is about 80 to about 200 nm.

The invention provides a synthetic polymer nanovesicle encapsulating either PEG-PEI-NAA polyplex or NAA alone, wherein the nanovesicle optionally comprises surface modifications and attached moieties for delivery of NAA to tissues and cells, further wherein the functionalization moiety of the nanovesicle comprises a protein selected from the group consisting of tumor-markers, integrins, cell surface receptors, transmembrane proteins, ion channels, membrane transport protein, enzymes, antibodies, chimeric proteins, and combinations thereof.

The invention provides a method of making a synthetic polymer nanovesicle comprising a synthetic polymer with encapsulated PEG-PEI-NAA polyplex or NAA alone, wherein the synthetic polymer is functionalized with surface coatings and moieties comprising providing a synthetic polymer nanovesicle wherein the synthetic polymer is functionalized with surface coatings and moieties, providing PEG-PEI-NAA polyplex or NAA alone, encapsulating the PEG-PEI-NAA polyplex or NAA alone in the synthetic polymer nanovesicle further wherein the nanovesicle is biologically degradable, chemically degradable, or both biologically and chemically degradable.

The invention provides a PEG-PEI-Nucleic Acid Agent (NAA) polyplex comprising a PEG-PEI copolymer, optionally comprising one or more functionalization moieties, and a NAA, wherein the NAA is associated with the copolymer by electrostatic interactions, further comprising a synthetic polymer nanovesicle in the form of a microbubble encapsulating the PEG-PEI-NAA polyplex, wherein release of encapsulant from the microbubble is triggered by ultrasound, and further wherein the synthetic polymer is a member selected from the group consisting of poly(lactic acid) (PLA), poly(glycolic acid) (PGA), and poly(lactic-co-glycolic acid) (PLGA).

### BRIEF DESCRIPTION OF SEVERAL VIEWS OF THE DRAWINGS

FIG. 1. Structural and physiochemical properties of non-limiting variants of PEG-PEI-NAA polyplexes used in the invention. FIG. 1a is a schematic representation of polyplex structural properties. FIG. 1b is a bar chart representation of the hydrodynamic diameter of the two PEG-PEI-NAA polyplexes shown in panel a, as measured by dynamic light scattering over a range of N:P values. FIG. 1c is a bar chart representation of the polyplex surface charge measured by zeta potential over a range of N:P values. FIG. 1d is a chart of the stability of PEG-PEI-NAA polyplexes evaluated by a polyanion competition assay. Details of these data are found in our recent publication (Glodde, M et. al. (2006) Biomacromolecules. V7(1). 347-356).

FIG. 2. Induction of dystrophin expression in mdx mice 3 weeks after intramuscular injection of PEG-PEI-NAA polyplexes. Dystrophin immunolabeling of transverse sections (Hoechst dye counterstained) from TA muscles at 2 different magnifications from the following groups: normal age-matched controls, mdx untreated, mdx injected with AO alone (20 μg), and mdx injected with PEI2K(PEG550)10-AO (20 μg AO).

FIG. 3. Induction of dystrophin expression in mdx mice 3 weeks after intramuscular injection of PEG-PEI-NAA polyplexes showing dystrophin-positive fibers were broadly, but not uniformly, distributed throughout the muscle cross-section. FIG. 3a is a micrograph of dystrophin immunolabeling of an entire transverse section of TA muscle 3 wks after intramuscular injection of 20 μg AO complexed with PEI2K(PEG550)10. FIG. 3b shows high magnification images of four different regions of the transverse section (labeled a-d).

FIG. 4. Quantitative evaluation of dystrophin-positive fibers in TA muscles of mdx mice at 3 weeks after intramuscular injection of either 20 pg of AO alone (N=4) or 20 μg of AO complexed with the following polymers: PEI2K(PEG550)10 (N=8), PEI25K(PEG5K)25 (N=6), PEI25K(PEG5K)50 (N=5), and pluronic F127 (N=4). Untreated mdx muscles (N=4) contained a small number of “so called” revertant fibers. All groups showed significantly fewer dystrophin positive fibers than injections of PEI2K(PEG550)10 (ANOVA; p<0.05).

FIG. 5. Western analysis of dystrophin expression in TA muscles of mdx mice at 3 and 9 wks after intramuscular injections of AO complexed with low MW PEG-PEI copolymers. Both images show blots of dystrophin (top) and vinculin (loading control; bottom) obtained from the same gel. Normal and mdx control muscle samples are shown. All samples were prepared by the extraction of thick (60 μm) transverse cryosections. Samples are as indicated in the figure key. Dystrophin expression as a percent of normal is indicated in parentheses below each lane.

FIG. 6. Efficient induction of dystrophin expression in mdx TA muscles at 9 wks after a single intramuscular injection of 20 μg of AO complexed with PEI2K(PEG550)10 copolymer. FIG. 6a shows micrographs of dystrophin immunolabeling transverse sections of TA muscles from normal, mdx control, and polyplex-treated muscles. FIG. 6b shows H&E staining of sections serial to those in the top panel.

FIG. 7. Dystrophin immunolabeling of TA muscle from mdx mouse at 9 and 16 wks after intramuscular injection with GNP-PEI2K(PEG550)₁₀-AO polyplex.

FIG. 8. Western analysis of dystrophin expression in mdx mice. Blots show dystrophin (top) and vinculin (loading control; bottom) obtained from the same gel. Dystrophin expression as a percent of normal is indicated below each lane. Lane IDs: (1) normal TA, (2) mdx TA at 9 wks after i.m. injection of GNP-PEI2K(PEG550)10-AO, (3) mdx TA at 2 wks after 6th tail vein injection of PEI2K(PEG5K)10-AO, (4-5) mdx gastroc at 2 wks after 6th tail vein injection of PEI2K(PEG5K)10-AO.

FIG. 9. FIG. 9a is a bar chart representation of the diameter of PLGA nanovesicles loaded with high MW PEI25K(PEG5K)10-AO determined by DLS. FIG. 9b is a bar chart representation of the encapsulation efficiency of PEG-PEI-AO and AO alone into PLGA nanovesicles. FIG. 9c is a micrograph of dystrophin immunolabeling of TA muscle of mdx mouse 3 wks after IM injection of PLGA encapsulated with high Mw PEI25K(PEG5K)10-AO. The image shows 4 individual regions of a single transverse section.

### DETAILED DESCRIPTION OF PREFERRED EMBODIMENTS OF THE INVENTION

To date, the major limitation to the in vivo usage of small nucleic acid agents such as antisense oligonucleotides has been inefficient and non-selective delivery to target cells. This invention will markedly improve the in vivo delivery of small nucleic acid agents to target tissues and cells with minimal cytotoxic effects or immune response. With this invention, small nucleic acid agents can be efficiently delivered locally or systemically to specific tissues including muscle cells (skeletal, cardiac, and smooth), neurons (including brain, spinal, and peripheral neurons), endothelial cells, cancerous tumors, and most, if not all, other organs of the body.

This invention encompasses the preparation and use of adaptable and versatile compounds that combine small nucleic acid agents with functionalized carriers such as nanopolymers and nanovesicles for improved delivery to tissues and cells. The carrier-NAA compounds are engineered with improved functionality including: improved transfection capacity, enhanced stability, cell/tissue specific targeting, and controlled release properties. The compositions are specifically formulated for in vivo usage.

The following abbreviations are used in the disclosure:
AO antisense oligoribonucleotide
PEI polyethylenimine
PEG polyethylene glycol
PEGylated PEI PEG chains grafted onto PEI
PEG-PEI copolymer PEI grafted with PEG chains
NAA nucleic acid agent
polyplex NAA complexed with PEG-PEI copolymer
N:P ratio molar ratio of PEI amines (N) to oligonucleotide phosphates (P) in a polyplex
MW molecular weight
Da dalton
GNP gold nanoparticle
PLGA poly(lactide-co-glycolide)
NLS nuclear localization signal

PEG-PEI Copolymers: Carriers for In Vivo Delivery of Nucleic Acid Agents

Polyethylenimine (PEI)

The amine-rich cationic polymer polyethylenimine (PEI) is an efficient nucleotide carrier that binds to the negatively-charged phosphate backbone of nucleotides and negatively-charged elements of cell membranes, facilitating endocytotic uptake of PEI-nucleotide complexes into cells (Bieber, T et. al. (2002) J. Control Release V82.441-454.; Boussif, O et. al. (1995) Proc. Natl. Acad. Sci. U.S.A V92. 7297-7301.; Godbey, W T et. al. (1999) Proc. Natl. Acad. Sci. U.S.A V96. 5177-5181.; Kichler, A (2004) J. Gene Med. V6 Suppl 1. S3-10.; Petersen, H et. al. (2002) Bioconjug. Chem. V13.845-854.; Petersen, H et. al. (2002) Macromolecules V35. 6867-6874.; Suh, J et. al. (2003) Proc. Natl. Acad. Sci. U.S.A V 100. 3878-3882.; Thomas, M et. al. (2003) Appl. Microbiol. Biotechnol. V62. 27-34). Protonation of the amine groups on PEI within endosomal compartments (the “so called proton sponge effect) is thought to cause osmotic lysis, and release of the endosomal contents (Akinc, A et. al. (2004) J. Gene Med. V; Boussif, O et. al. (1995) Proc. Natl. Acad. Sci. U.S.A V92. 7297-7301.; Hara-Chikuma, M et. al. (2004) Journal of Biological Chemistry V; Sonawane, N D et. al. (2003) Journal of Biological Chemistry V278. 44826-44831.; Thomas, M et. al. (2003) Appl. Microbiol. Biotechnol. V62. 27-34). Once released, AO may enter the nucleus by a process that likely requires dissociation from the PEG-PEI copolymer.

Previous studies of PEI-based transfection in skeletal muscle used high MW PEI without PEG, which was found to be relatively ineffective and very cytotoxic (Bremmer-Bout, M et. al. (2004) Mol. Ther. V10. 232-240.; Lu, Q L et. al. (2003) Gene Ther. V 10. 396-405). Based on these results it was concluded that cationic polymers are unsuitable for in vivo delivery of AO in skeletal muscle (Lu, Q L et. al. (2003) Nat. Med. V9. 1009-1014.; Lu, Q L et. al. (2003) Gene Ther. V10. 131-142). However, the PEI-AO particles used in these previous studies undoubtedly had very high positive surface charges, because they did not contain PEG, which is known to provide steric shielding of the PEI surface charge. In addition, dispersion of the highly-charged particles after intramuscular injection is probably severely hindered by charge interactions between the PEI and negatively-charged elements within the extracellular environment. Therefore, it is not surprising that these previous muscle transfection studies with non-PEGylated PEI produced unsatisfactory results.

Polyethylene Glycol (PEG)

The functionality of PEI as a nucleotide carrier is significantly improved by incorporating the nonionic linear polymer polyethylene glycol (PEG) into PEG-PEI copolymers (Kichler, A (2004) J. Gene Med. V6 Suppl 1. S3-10.; Petersen, H et. al. (2002) Macromolecules V35. 6867-6874.; Sung, S J et. al. (2003) Biol. Pharm. Bull. V26. 492-500). Complexation of PEG-PEI copolymers with small nucleic acid agents produces particles with a core-shell structure, whereby PEI and the electrostatically-coupled nucleic acid agent are oriented toward the particle core and the PEG chains form a brushlike corona, although the precise nature of this arrangement remains an open question. In general, PEG provides polyplexes with improved solubility, lower surface charge, diminished aggregation, lower cytotoxicity, and decreased opsonization with serum proteins in the bloodstream. However, these desirable properties may come at a cost of lower transfection efficiency due to both reduced membrane interaction and less efficient endosomal escape. The precise function of PEGylation may depend on many factors including PEG molecular weight, PEI molecular weight, stoichiometry of PEG grafting, type of nucleic acid agent, and environment.

Overall, by varying the MW and the arrangement of PEG and PEI, PEG-PEI copolymers represent a flexible nucleotide delivery system with controllable size and adjustable unpackaging properties. Although PEG-PET copolymers have been primarily used for plasmid delivery, other studies have provided strong evidence that PEGylated PEI can be adapted to function as an effective carrier for cellular delivery of small oligonucleotides (Brus, C et. al. (2004) Bioconjug. Chem. V15. 677-684.; Fischer, D et. al. (2004) Drug Metab Dispos. V32.983-992.; Jeong, J H et. al. (2003) J. Control Release V93.183-191.; Kunath, K et. al. (2002) Pharm. Res. V19.810-817.; Schiffelers, R M et. al. (2004) Nucleic Acids Res. V32. e149-; Vinogradov, S et. al. (1999) Bioconjug. Chem. V10. 851-860.; Vinogradov, S V et. al. (1998) Bioconjug. Chem. V9.805-812.; Vinogradov, S V et. al. (2004) Bioconjug. Chem. V15. 50-60).

The Examples demonstrate that specific formulations of PEG-PEI copolymers function as effective NAA carriers, resulting in broad distribution of dystrophin-positive fibers (resulting from the action of the NAA) following intramuscular and systemic injections, without any indication of cytotoxicity. The examples document that functionalized PEG-PEI-NAA polyplexes show improved in vivo induction of gene expression in mammals.

Nucleic Acid Agent (NAA)

As used herein, the term ‘nucleic acid agent’ includes any nucleic acid molecules, especially antisense oligoribonucleotides, ribozymes, aptamers, oligodeoxynucleotides (ODN), small nuclear RNA (snRNA), U7-snRNA, short interfering RNA (siRNA), short hairpin RNA (shRNA), peptide nucleic acid (PNA), nucleoside 5′ triphosphates, and combinations thereof. Examples of compounds falling within this group include DNA and RNA for transfection. Included within the group of ribozymes are external guide sequences for directing cleavage of a substrate RNA by RNase P. Nucleotide molecules may be RNA, DNA, or modified nucleic acid molecules including derivatives or modified nucleotides which enhance stability.

Ribonucleic acid (RNA) molecules can serve not only as carriers of genetic information, for example, genomic retroviral RNA and messenger RNA (mRNA) molecules and as structures essential for protein synthesis, for example, transfer RNA (tRNA) and ribosomal RNA (rRNA) molecules, but also as enzymes which specifically cleave nucleic acid molecules. Such catalytic RNA molecules are called ribozymes.

Chemical modifications can be made which greatly enhance the nuclease resistance of an oligonucleotide without compromising its biological function. For example, one or more of the bases can be replaced by 2′ methoxy ribonucleotides, phosphorothioate deoxyribonucleotides, or phosphorothioate ribonucleotides, 2′O-methyl, phosphorthioate, 2′MEO, phosphodiester, and combinations thereof, using available nucleic acid synthesis methods (see for example, Offensperger et. al., EMBO J., 12:1257-1262 (1993); WO 93/01286 by Rosenberg et al.; Agrawal et al., Proc. Natl. Acad. Sci. USA,. 85:7079-7083 (1988); Sarin et al., Proc. Natl. Acad. Sci. USA, 85:7448-7794 (1989); Shaw et al., Nucleic Acids Res, 19:747-750 (1991); incorporated herein by reference).

Another class of chemical modifications is modification of the 2‘OH group of a nucleotide’s ribose moiety, which has been shown to be critical for the activity of the various intracellular and extracellular nucleases. Typical 2′ modifications are the synthesis of 2′-O-Methyl oligonucleotides (Paolella et al., EMBO J., 11:1913-1919, 1992) and 2′-fluoro and 2′-amino-oligonucleotides (Pieken, et al., Science, 253:314-317 (1991); Heidenreich and Eckstein, J. Biol. Chem., 267:1904-1909 (1992)).

The Anti-N1 2′OMeAO (5′-AUUCACUUUCAUAAUGCUGG-3′) previously described by Singh et al. has been used (Singh, N K et. al. (2006) Mol. Cell Biol. V26. 1333-1346). This AO blocks a splice inhibition motif within intron 7 of hSMN2 and has been shown to cause “splicing in” of exon 7, resulting in a switch from SMN2-delta7 to mostly full-length SMN2 protein.

The h51 AON 2′ OMeAO (5′-UCAAGGAAGAUGGCAUUUCU-3′) was previously described (Aartsma-Rus, A et. al. (2002) Neuromuscul. Disord. V12 Suppl 1. S71-S77). This AO causes skipping of exon 51 of the human dystrophin gene and has been shown to facilitate expression of nearly full-length dystrophin.

PEG-PEI-NAA Polyplexes

The invention provides a family of low and high MW PEG-PEI copolymers. Detailed analysis of the physiochemical properties of the resultant PEG-PEI-NAA polyplexes was performed (Glodde, M et. al. (2006) Biomacromolecules. V7(1). 347-356). By utilizing copolymers that covered a range of PEI MW, PEG MW, and stoichiometry of PEG grafting, a dynamic range of polyplex size, surface charge, and stability was revealed. Each of these properties are in-turn expected to influence polyplex transfection capacity.

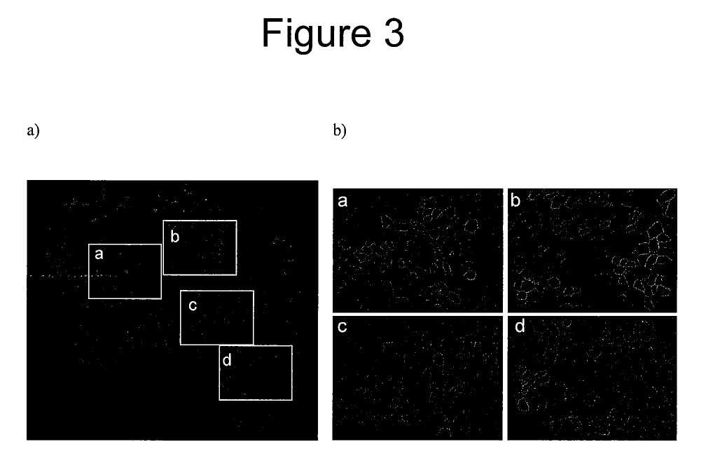

The MW of PEG was found to be the main determinant of polyplex size, via its influence on particle aggregation. Dynamic light scattering (DLS) measurements showed that when PEG5K was grafted to either PEI2K or PEI25K, polyplex diameter was extremely small (˜10 nm) with no apparent aggregation (see FIG. 3 in reference (Glodde, M et. al. (2006) Biomacromolecules. V7(1). 347-356)). In contrast, when PEG550 was grafted to PEI2K, polyplexes appeared as much larger aggregate particles (˜250 nm). As expected zeta potential measurements showed that surface charge of the low MW PEI2K polyplexes was significantly lower than high MW PEI25K-based polyplexes, when evaluated at equivalent N:P ratios (see FIG. 3 in reference (Glodde, M et. al. (2006) Biomacromolecules. V7(1). 347-356)).

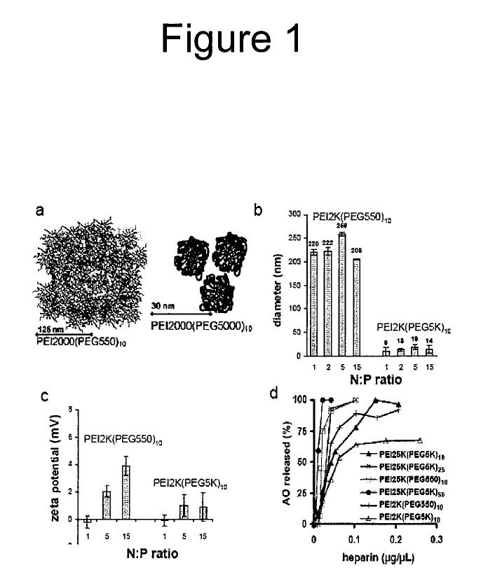

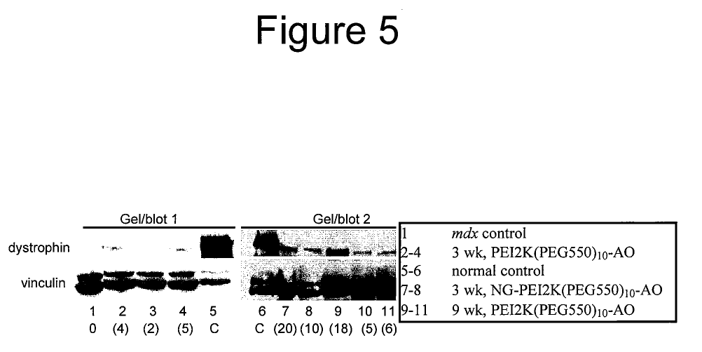

The association-dissociation behavior of PEG-PEI-NAA polyplexes is a critical aspect that governs their transfection efficiency (Dass, C R (2002) J. Pharm. Pharmacol. V54. 3-27.; Dias, N et. al. (2002) Mol. Cancer Ther. V1. 347-355.; Hughes, M D et. al. (2001) Drug Discov. Today V6. 303-315.; Merdan, T et. al. (2002) Adv. Drug Deliv. Rev. V54.715-758.; Roth, C M et. al. (2004) Annu. Rev. Biomed. Eng V6. 397-426.; Shi, F et. al. (2004) J. Control Release V97.189-209.; Wiethoff, C M et. al. (2003) J. Pharm. Sci. V92. 203-217). In a useful polyplex delivery system, the electrostatic charge association between the AO and copolymer must be strong enough to promote cellular uptake, but not too strong as to prohibit release of “free AO”, so that AO can be translocated to myonuclei. A polyanion competition assay was used to assess the relative stability, or association-dissociation dynamics, of the various polyplexes (FIG. 1d; see also FIGS. 5 & 6 in (Glodde, M et. al. (2006) Biomacromolecules. V7(1). 347-356)). Surprisingly, PEI2K-based polyplexes proved to be substantially more stable than several of the PEI25K-based polyplexes. The stability of the PEI2K-based polyplexes is one of their salient features that explain their high transfection capacity in vivo.

The invention provides PEG-PEI-NAA polyplexes. Non-limiting examples of three basic types of PEG-PEI-NAA polyplexes, each of which have unique properties and may be preferred in specific drug delivery modalities and applications are Low MW PEI2K with covalently grafted PEG5K (copolymer=PEI2K(PEG5K)₁₀), Low MW PEI2K with covalently grafted PEG550 (copolymer=PEI2K(PEG550)₁₀), and High MW PEI25K with covalently grafted PEG5K (copolymer=PEI25K(PEG5K)₁₀).

Low MW PEI2K with covalently grafted PEG5K (copolymer=PEI2K(PEG5K)₁₀)

When the PEI2K(PEG5K)₁₀ copolymer is complexed with NAA, nanoparticulates are formed with desirable properties for in vivo delivery of NAA to target tissues and cells. The salient features of the resultant polyplexes include extreme stability, nanosized and non-aggregated particles, and low surface charge. The high stability of these polyplexes was not described in the prior art and could not be predicted from prior studies. The high stability is the key feature of these polyplexes. The preferred formulation was derived from systematic evaluation of the physiochemical and biological structure-function analysis (this also applies to the other polyplex formulations below). The unifying concept is that PEGylation with long PEG chains (i.e., PEG5K) provides sufficient steric repulsive forces that deters aggregation. This is true whether the PEI is the low or high MW variety.

Low MW PEI2K with covalently grafted PEG550 (copolymer=PEI2K(PEG550)₁₀)

When the PEI2K(PEG550)₁₀ copolymer is complexed with NAA, nanoparticulates are formed with desirable properties for in vivo delivery of NAA to target tissues and cells. The salient features of the resultant polyplexes include extreme stability, aggregated particles with high carrying capacity (ie., high payload of NAA per endocytotic event), and low surface charge. The aggregate polyplexes may be especially useful, as they likely contain more NAA per particle, and therefore more NAA may be taken up into cells per endocytotic event. The short PEG chains provide adequate shielding of surface charge, but do not deter aggregation. The aggregate structure may also resist degradation of NAA. The prior art has not discussed that aggregate structures may be preferable to non-aggregated compounds for specific in vivo applications.

High MW PEI25K with covalently grafted PEG5K (copolymer=PEI25K(PEG5K)₁₀)

Due mainly to their substantial surface charge, high MW PEG-PEI-NAA polyplexes are inherently more potent than their low MW counterparts (Kursa, M et. al. (2003) Bioconjug. Chem. V14. 222-231.; Ogris, M et. al. (1999) Gene Ther. V6.595-605.; Ogris, M et. al. (2001) AAPS. Pharm Sci. V3. E21-; Ogris, M et. al. (2003) J. Control Release V91. 173-181). We previously showed that when N:P ratio and PEG grafting were optimized, high MW PEI25K(PEG5K)₁₀ copolymers formed polyplexes with oligonucleotides that were very stable, small (˜15 nm), non-aggregated, and had a relatively high positive surface charge (see FIG. 3 in (Glodde, M et. al. (2006) Biomacromolecules. V7(1). 347-356)). These high MW polyplexes provided effective AO delivery to myonuclei of isolated mature skeletal muscle fibers (Sirsi, S R et. al. (2005) Hum. Gene Ther. V16 (11). 1307-1317). As expected, their in vitro transfection capacity was substantially greater than low MW polyplexes (data not shown). Despite the relatively high surface charge, the toxicity of PEI25K(PEG5K)₁₀-oligonucleotide polyplexes was negligible due to optimized PEGylation. Indeed the level of PEG grafting and N:P ratio that yielded both high potency and negligible toxicity was empirically determined. The salient features of PEI25K(PEG5K)₁₀-NAA polyplexes include excellent stability, nanosized and non-aggregated particles, and relatively high surface charge. Because of the high surface charge, this intrinsically potent compound must be encapsulated into a delivery vehicle for in vivo usage. In this invention PEI25K(PEG5K)₁₀-NAA polyplexes will be encapsulated in functionalized PLGA nanovesicles and PLGA microbubbles that shield the high surface charge and provide favorable biodistribution and controlled delivery of NAA to target tissues.

It should be noted that PEI25K(PEG5K)₁₀-NAA polyplexes (and similar) are also idea for in vitro transfection without encapsulation. For in vitro (cell culture) applications, the high surface charge does not impede distribution to target cells, so there is no obvious need to encapsulate. However, further functionalization of these polyplexes as described below for in vivo usage (e.g., GNP, TAT, etc) is also applicable to improved functionality for in vitro applications.

Functionalized PEG-PEI Copolymers

The invention provides that copolymers will be further functionalized for enhanced delivery of polyplexes to target tissues and enhanced cellular uptake. Addition of functional groups to the copolymers will improve polyplex stability, improve polyplex biodistribution, improve polyplex tissue delivery, improve polyplex and/or cellular uptake, and combinations thereof. Non-limiting examples of functional groups provided by the invention are gold nanoparticles (GNP), TAT-PTD and derivatives thereof, ApoE, albumin, antibody, antibody fragment, magnetic nanoparticle, iron oxide, transferrin, AAV tropism fragment, and combinations thereof.

Gold Nanoparticles

Colloidal gold is a versatile agent for a wide variety of biomedical applications, including the delivery of genes and drugs (Hainfeld, J F et. al. (2000) J. Histochem. Cytochem. V48. 471-480). Gold nanoparticles (GNP) have previously been shown to improve cellular uptake and biocompatibility of polymeric nucleotide carriers (Hainfeld, J F et. al. (2000) J. Histochem. Cytochem. V48. 471-480.; Thomas, M et. al. (2003) Proc. Natl. Acad. Sci. U.S.A V100. 9138-9143). Internalization of gold nanoparticles into various cell types has been demonstrated (Kaisto, T et. al. (1999) Exp. Cell Res. V253.551-560.; Shukla, R et. al. (2005) Langmuir V21. 10644-10654.; Thomas, M et. al. (2003) Proc. Natl. Acad. Sci. U.S.A V100. 9138-9143). In addition to facilitating cellular uptake, gold nanoparticles are inherently non-cytotoxic and have been shown to reduce the production of reactive oxygen and nitrite species, and prevent stimulation of proinflammatory cytokines (Shukla, R et. al. (2005) Langmuir V21. 10644-10654). Previous studies have also shown that coating of polymers with gold particles significantly reduced platelet and monocyte activation and reduced bacterial adhesion, generally improving the biocompatibility of the polymers (Hsu, S H et. al. (2006) J. Biomed. Mater. Res. A V).

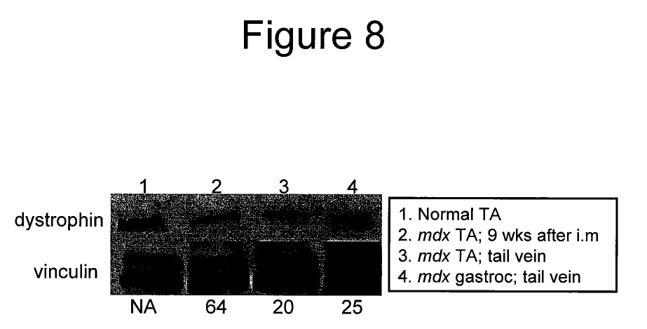

This information indicates that conjugation of GNPs to PEG-PEI copolymers will improve cellular uptake of polyplexes and reduce cytotoxicity. Our recent experiments showed that IM injections of GNP-PEG-PEI-NAA polyplexes resulted in substantially higher levels of dystrophin expression than with un-conjugated copolymers with peak levels reaching 65% of normal levels (FIGS. 5 and 8). Among other uses, the GNP-PEG-PEI carriers will provide effective delivery of NAA to body musculature after intravenous and intramuscular delivery, and also to brain and spinal cord after direct injection into CSF. These compounds may also provide effective delivery of AO to CNS after intravenous injections.

TAT Protein Transduction Domain

The protein transduction domain (PTD) of HIV-TAT (trans activator of transcription) consists of an arginine-rich 11 aa peptide, and has been exploited as a carrier for the in vivo delivery of a variety of compounds. Since the discovery that only 11 amino acids of TAT are required for transduction, the use of TAT PTD has increased significantly (Ho, A et. al. (2001) Cancer Res. V61. 474-477.; Nagahara, H et. al. (1998) Nat. Med. V4. 1449-1452.; Toro, A et. al. (2006) J. Clin. Invest V116. 2717-2726). It is well established that fusion complexes of TAT-PTD-cargo are able to circulate in the bloodstream, escape the microvasculature and undergo cellular uptake in a variety of tissues including skeletal muscles, brain cells, and the myocardium (Brooks, H et. al. (2005) Adv. Drug Deliv. Rev. V57.559-577.; Moulton, H M et. al. (2003) Antisense Nucleic Acid Drug Dev. V13. 31-43). Nuclear localization sequences within TAT-PTD can also promote nuclear uptake of the fusion complexes (Toro, A et. al. (2006) J. Clin. Invest V116. 2717-2726). TAT-PTD will be conjugated to PEG-PEI copolymers and complexed with NAA to form TAT-PEG-PEI-NAA polyplexes. TAT-PEG-PEI-NAA polyplexes will significantly improve delivery of NAA to target cells after local and systemic delivery.

ApoE

PEG-PEI copolymers will be conjugated with apolipoprotein E (ApoE), a 34 kD low-density lipoprotein (LDL) binding protein. It was recently shown that ApoE facilitates the delivery of drugs by utilizing both endocytotic and transcytotic mechanisms of transport (Kreuter, J et. al. (2002) J. Drug Target V 10.317-325.; Kreuter, J (2004) J. Nanosci. Nanotechnol. V4.484-488.; Michaelis, K et. al. (2006) J. Pharmacol. Exp. Ther. V317. 1246-1253). These processes likely involve the LDLR, which is up-regulated on the surface of endothelial cells of the microvasculature and on the plasma membrane of various cell types including skeletal muscle fibers (Dehouck, B et. al. (1997) Journal of Cell Biology V138.877-889.; Dergunov, A D et. al. (2005) Biol. Chem. V386.441-452.; Lucarelli, M et. al. (2002) FEBS Lett. V522.19-23.; Ribalta, J et. al. (2003) Curr. Opin. Clin. Nutr. Metab Care V6.177-187.). LDLR-ligand complexes are taken into clathrin coated pits and delivered to endosomes where the low pH environment triggers release of bound particles (Beglova, N et. al. (2005) Trends Biochem. Sci. V30.309-317.; Brown, M S et. al. (1986) Science V232. 34-47). ApoE has been shown to traffic to late endosomes and lysosomes (Kaisto, T et. al. (1999) Exp. Cell Res. V253. 551-560). Therefore, conjugation with ApoE will enhance the delivery of PEG-PEI-NAA polyplexes across the microvasculature and enhance cellular uptake into target cells. The relatively small size and high solubility of ApoE2 makes it ideally suited for conjugation to the PEG-PEI copolymers. The ApoE2 isoform is not associated with any known cytotoxicity, and is even thought to have anti-inflammatory properties (Dodart, J C et. al. (2005) Proc. Natl. Acad. Sci. U.S.A V102. 1211-1216).

Albumin

Conjugation albumin (ALB; a 66 kDa serum protein) to PEG-PEI copolymers will improve cellular uptake of polyplexes into target cells and increase transport across the microvasculature. Albumin conjugation to ligands and nanoparticles has been demonstrated to improve the circulation half-life of drugs injected into the bloodstream (Dennis, M S et. al. (2002) Journal of Biological Chemistry V277. 35035-35043.; Robinson, D M et. al. (2006) Drugs V66. 941-948.) and has been used in drug delivery applications to facilitate transcytosis across the capillary endothelium to target cells (Gradishar, W J (2006) Expert. Opin. Pharmacother. V7. 1041-1053.; Hillaireau, H et. al. (2006) J. Nanosci. Nanotechnol. V6. 2608-2617). Albumin transcytosis and cellular uptake appear to be mediated by caveolin-dependent processes (Cohen, A W et. al. (2004) Physiol Rev. V84.1341-1379.; Vogel, S M et. al. (2001) Am. J. Physiol Lung Cell Mol. Physiol V281. L1512-L1522). Among other actions and uses, conjugation of recombinant albumin to PEG-PEI copolymers will increase polyplex transcytosis and improve cellular uptake of polyplexes. Although albumin has been utilized previously for enhanced delivery of drugs, conjugation of albumin to cationic carriers for improved delivery of NAA is a novel strategy.

Antibodies

Antibodies refers to single chain, two-chain, and multi-chain proteins and glycoproteins belonging to the classes of polyclonal, monoclonal, chimeric, and hetero immunoglobulins (monoclonal antibodies being preferred); it also includes synthetic and genetically engineered variants of these immunoglobulins. “Antibody fragment” includes Fab, Fab′, F(ab′)2, and Fv fragments, as well as any portion of an antibody having specificity toward a desired target epitope or epitopes. A humanized antibody is an antibody derived from a non-human antibody, typically murine, that retains or substantially retains the antigen-binding properties of the parent antibody but which is less immunogenic in humans (Jones et al., Morrison et al., Proc. Natl. Acad. Sci. USA, 81:6851-6855 (1984); Morrison and Oi, Adv. Immunol., 44:65-92 (1988); Verhoeyen et al., Science, 239:1534-1536 (1988); Padlan, Molec. Immun., 28:489-498 (1991); Padlan, Molec. Immun., 31(3):169-217 (1994)).

Magnetic Nanoparticles

Magnetic nanoparticles consist of a ferri- or ferromagnetic material and have biologically active and/or therapeutically effective envelope layers. On the one hand, they are able to permeate the cell membrane of cells and, on the other hand, to attach with high specificity to targets present in the intracellular region of malignant cells. Generally, the size of the nanoparticles according to the invention is from 2 to 100 nm. The nanoparticles have out-standing properties with respect to their capability of permeating cell membranes and their improved physical compatibility. Although having a relatively low magnetic moment as a result of their small volume, intracellular particle agglomeration caused by binding to intracellular target biomacromolecules results in an augmented concentration with increased magnetic moment of the malignant cells to be removed, thereby promoting magnetic separation. The nanoparticles may prepared from iron oxide particles. (U.S. Pat. No. 6,514,481).

Transferrin

Transferrin is the protein that transports iron in human and animal plasma, in which its concentration is approximately 2.5 g/l. This major function of transferrin derives from its ability to specifically bind trivalent iron. Once iron is resorbed into the small intestine or picked up by the iron-storage protein ferritin, it is transported in the trivalent form to other tissues. (U.S. Pat. No. 5,041,537).

AAV Tropism Fragment

Adeno-associated virus (AAV) tropism fragment is a segment of an AAV vector that can facilitate cell entry and/or nuclear localization of attached ligands. The fragment of AVV is expected to be unique to the various AAV serotypes and may therefore show unique and cell type-specific properties in terms of cellular uptake.

Methods of Preparation of PEG-PEI Copolymers and Functionalized Derivatives

The methods of PEG-PEI copolymer synthesis are known in the art, and both low and high MW variations of PEG-PEI copolymers have been described (Glodde, M et. al. (2006) Biomacromolecules. V7(1). 347-356.; Petersen, H et. al. (2002) Macromolecules V35. 6867-6874).

The methods of conjugating surface moieties and ligands to PEG-PEI copolymers and PLGA nanovesicles are known in the art. Example 7 describes coupling of TAT-PTD to PEG-PEI copolymers. In another embodiment recombinant serum albumin will be conjugated to PEG-PEI copolymers using a zero-spacer EDC linkage, coupled through amine reactive sulfo-NHS. Although this protocol is generally used for protein-protein coupling, adaptation permits coupling of albumin carboxyl groups to amine groups within PEI.

PEG-PEI-NAA Polyplex Formulations

Table 1 shows non-limiting examples of PEG-PEI copolymers to be utilized in the invention. The rationale for choosing each of the specified copolymers and variations are detailed below. In most cases conjugation of specific moieties to PEG-PEI will not only increase cellular uptake into cells, but may also enhance delivery across the microvasculature after systemic delivery.

Non-limiting examples of PEG-PEI copolymers include PEI2K(PEG550)₁₀, PEI2K(PEG5K)₁₀, GNP-PEI2K(PEG550)₁₀, GNP-PEI2K(PEG5K)₁₀, PEI2K(PEG550)₅(hexyl)₅, PEI2K(PEG5K)₅(hexyl)₅, ALB-PEI2K(PEG550)₁₀, ALB-PEI2K(PEG5K)₁₀, ApoE-PEI2K(PEG550)₁₀, ApoE-PEI2K(PEG5K)₁₀, TAT-PEI2K(PEG550)₁₀, TAT-PEI2K(PEG5K)₁₀ The nomenclature used for copolymers indicates the MW (in daltons) of PEI and PEG, along with the number of PEG chains grafted per PEI (shown as subscript). For example the copolymer PEI2K(PEG550)₁₀ has 10 PEG chains of 550 Da grafted to PEI2K. The abbreviations for conjugates are as follows: gold nanoparticles (GNP), albumin (ALB), apolipoprotein (ApoE), and HIV-TAT protein transduction domain (TAT).

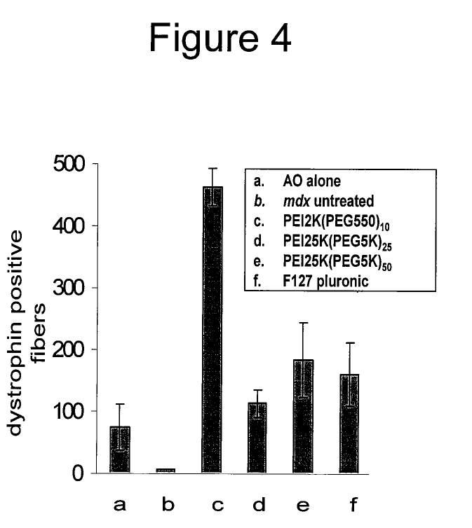

1. PEI2K(PEG550)₁₀—This copolymer forms polyplexes with NAA that are relatively large (250 nm), but very stable aggregate structures, with low surface charge (FIG. 1). These polyplexes outperformed two different high MW PEI25K-based copolymers and the commercial polymer F-127 in terms of number of dystrophin-positive fibers at 3 wks after intramuscular injection (FIG. 4). Pilot studies suggest that dystrophin expression with these polyplexes increased substantially between 3 and 9 wks post-transfection (FIG. 5).

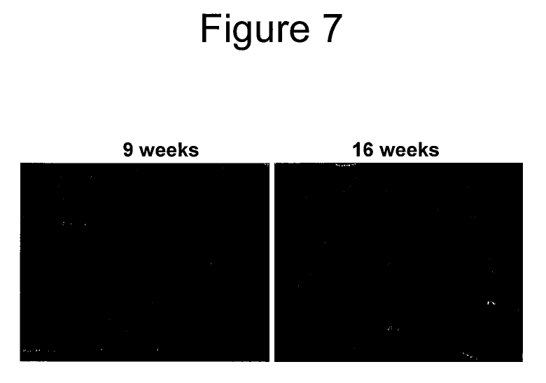

2. PEI2K(PEG5K)₁₀—This copolymer when complexed with AO forms tiny (10 nm) and extremely stable polyplexes, with very low surface charge (FIG. 1). We have shown that the long PEG chains (5 kDa) in this copolymer impart steric repulsive forces that deter aggregation; explaining why tiny particles are formed when complexed with AO (Glodde, M et. al. (2006) Biomacromolecules. V7(1). 347-356). Inspection of muscles at 3 weeks after intramuscular injection of these polyplexes showed significant induction of dystrophin expression (FIG. 7). Because of their low surface charge and extreme stability these polyplexes may be especially well-suited for delivery though the bloodstream.

3. GNP-PEI2K(PEG550)₁₀—Experiments showed that single IM injections of NG-PEI2K(PEG550)₁₀-AO polyplexes resulted in substantially higher levels of dystrophin expression than with the un-conjugated copolymer at 3 wks after injection (FIG. 5), and showed no overt signs of cytotoxicity (FIG. 7). Experiments also showed that dystrophin expression increased between 3 and 9 wks post-transfection, with peak levels reaching 65% of normal levels (FIG. 8).

4. GNP-PEI2K(PEG5K)₁₀—Following the same line of reasoning as for GNP-PEI2K(PEG550)₁₀, GNP conjugation will produce a significant improvement in potency, relative to the un-conjugated copolymer.

5-6. PEI2K(PEG550)₅(hexyl)₅ and PEI2K(PEG5K)₅(hexyl)₅—The addition of hexyl chains to PEG-PEI copolymers will increase the hydrophobicity of the resultant polyplexes. By increasing hydrophobicity, interactions between the polyplexes and the muscle cell membrane may be increased, resulting in higher transfection efficiency. The invention provides variants of PEI2K(PEG550)₁₀ and PEI2K(PEG5K)₁₀ copolymers, where about 50% of the PEG chains are replaced with hexyl chains.

7-8. ALB-PEI2K(PEG550)₁₀ and ALB-PEI2K(PEG5K)₁₀—Conjugation of recombinant albumin (ALB; a 66 kDa serum protein) to PEG-PEI copolymers will improve cellular uptake of polyplexes and increase transport from the bloodstream to the tissue interstitium. Albumin was described previously.

9-10. ApoE-PEI2K(PEG550)₁₀ and ApoE-PEI2K(PEG5K)₁₀-PEG-PEI copolymers will be conjugated with apolipoprotein E (ApoE), a 34 kD low-density lipoprotein (LDL) binding protein described previously.

11-12. TAT-PEI2K(PEG550)₁₀ and TAT-PEI2K(PEG5K)₁₀—The TAT PTD was described previously. The TAT-PEG-PEI-NAA polyplexes will significantly improve delivery of NAA after both local and systemic delivery.

Method of Preparing PEG-PEI-NAA Polyplexes

The PEG-PEI-NAA polyplexes will be prepared by simple mixing of copolymer and NAA at a given nitrogen to phosphate N:P ratio using methods known in the art (Glodde, M et. al. (2006) Biomacromolecules. V7(1). 347-356). Additionally, ligands and moieties can be readily attached to copolymers using materials commonly available and using methods known in the art including NHS-PEOₙ-MAL bifunctional linker (Pierce) and EDC chemistry.

Functionalized Synthetic Polymer Nanovesicles for Delivery of Carrier-NAA Compounds

Due to its cationic nature there is an inherent tradeoff in the PEG-PEI-NAA delivery system between transfection capacity and biodistribution. On one hand the residual positive surface charge on PEG-PEI-NAA polyplexes is important for stimulating cellular uptake. On the other hand the positive surface charge acts to decrease circulation time in the blood and limits diffusional distribution in the tissue interstitium due to electrostatic interactions with negatively charged elements in the bloodsteam and interstitium. A useful alternative approach would be to encapsulate PEG-PEI-NAA polyplexes within inert and degradable vesicles to facilitate improved biodistribution and delivery to the interstitium of target tissues. This “encapsulation approach” may enhance NAA delivery by shielding polyplex surface charge and by protecting the NAA from enzymatic digestion.

Poly(lactic-co-glycolic acid) (PLGA) polymers are biodegradable and biocompatible compounds that have been approved by the Food and Drug Administration and utilized in a wide variety of drug delivery applications (Bala, I et. al. (2004) Crit. Rev. Ther. Drug Carrier Syst. V21. 387-422.; Panyam, J et. al. (2004) Curr. Drug Deliv. V1. 235-247). The versatility of PLGA vesicles stems from the fact the macromolecular properties can be controlled by varying the synthesis components and conditions, providing vesicles with a broad size range and variable release kinetics (Astete, C E et. al. (2006) J. Biomater. Sci. Polym. Ed V17. 247-289). Additionally, PLGA vesicles contain free carboxyl groups that are useful for attaching various moieties to the surface that can improve performance. Despite their proven utility in many drug delivery studies, PLGA vesicles have been used only sparingly for the delivery of polymer-NAA compounds, and this has been restricted to micron size vesicles (De Rosa, G et. al. (2002) J. Pharm. Sci. V91. 790-799.; De Rosa, G et. al. (2003) Int. J. Pharm. V254. 89-93.; DeRosa G. et. al. (2003) Biomacromolecules. V4.529-536.; Howard, K A et. al. (2004) Biochim. Biophys. Acta V1674. 149-157.; Moffatt, S et. al. (2006) Int. J. Pharm. V321.143-154).

PLGA slowly degrades by nonenzymatic hydrolysis of the ester backbone (Gopferich, A (1996) Biomaterials V17. 103-114.) into lactic acid (a natural body metabolite) and glycolic acid (excreted or degraded in the body), neither of which produces toxic effects (Brophy R M. Biodegradable polyester polymers as drug carriers. In: Swarbrick J, Boylan J C, editors. Encyclopedia of pharmaceutical technology. Vol. 2. New York: Marcel Dekker; 1990. p 1-25). Irrespective of external forces, the copolymer ratio of PLGA has an effect on the degradation profile. Microspheres made of a copolymer with high amounts of lactic acid will degrade slower than those made with a polymer of high glycolic acid content.

Degradation of PLGA results in oligomers, dimers, and monomers of the constituent lactic and glycolic acids. Several techniques, including gel permeation chromatography, gravimetry, scanning electron microscopy, differential scanning chromatography, and high-pressure liquid chromatography (HPLC), have been used to study the degradation of solid polymer microspheres. The quantification of the amount of lactic and glycolic acid monomers released over time provides an excellent comparative method for the study of the effects of fabrication method on the degradation pattern of microspheres (Giunchedi, P et. al. (1998) J. Control Release V56.53-62.; Park, T G (1995) Biomaterials V16. 1123-1130.; Vert, M et. al. (1994) Biomaterials V15. 1209-1213).

PLGA nanovesicles will be used to deliver carrier-NAA compounds, and preferably PEG-PEI-NAA polyplexes. PLGA nanovesicles will also be used to deliver NAA alone and carrier functionalized oligonucleotides (CFOs). Carrier-NAA and NAA compounds will be encapsulated in PLGA nanospheres of about 50-200 nm size. Encapsulated carrier-NAA compounds will include both low and high MW PEG-PEI-NAA polyplexes, and functionalized derivatives. PLGA-encapsulation will allow the polyplexes to circulate longer in the bloodstream by masking the polyplex surface charge, and by protecting the oligonucleotide from degradation.

Nanosized PLGA vesicles have better diffusional characteristics than their micron sized counterparts. Nanosized PLGA vesicles have also been shown to be endocytosed followed by endo-lysosomal escape (Panyam, J et. al. (2002) FASEB J. V16.1217-1226), which provides additional functionality to this NAA delivery system.

For reasons previously stated PLGA vesicles will be especially useful as a delivery vehicle for high MW PEG-PEI-NAA polyplexes, which are inherently more potent than their low MW counterparts (Kursa, M et. al. (2003) Bioconjug. Chem. V14. 222-231.; Ogris, M et. al. (1999) Gene Ther. V6.595-605.; Ogris, M et. al. (2001) AAPS. Pharm Sci. V3. E21-; Ogris, M et. al. (2003) J. Control Release V91. 173-181). We already showed that when N:P ratio and PEG grafting were correctly chosen, high MW PEI25K(PEG5K)₁₀ copolymers formed polyplexes with AO that were very stable, small (˜15 nm), and non-aggregated, and as expected had a substantial positive surface charge (FIG. 1 and see FIG. 3 in (Glodde, M et. al. (2006) Biomacromolecules. V7(1). 347-356)). We also showed that in vitro these high MW polyplexes provided effective AO delivery to myonuclei of mature skeletal muscle fibers (Sirsi, S R et. al. (2005) Hum. Gene Ther. V16 (11). 1307-1317). As expected, their in vitro transfection capacity was substantially greater than low MW polyplexes.

Although the carboxylic acid end groups on PLGA polymers are useful for attaching functional groups, they also create an overall negative surface charge on the resultant nanospheres. The negative surface charge is a potential barrier to effective delivery due to non-specific adsorption of positively charged serum proteins, prompting macrophage-mediated clearance from the blood stream (Muller, M et. al. (2003) J. Biomed. Mater. Res. A V66. 55-61). To overcome this problem, simple surface modifications of PLGA nanovesicles can be utilized to shield the surface charge and prolong circulation half-life. Surface coatings of synthetic polymer nanovesicles may also enhance delivery across the microvasculature and internalization into target cells.

Methods for Making PLGA Nanovesicles

In preferred embodiments, synthetic polymer nanovesicles with encapsulated carrier-NAA compounds will be surface coated with PEG, albumin, and GNP.

Surface coating of PLGA nanovesicles with PEG, albumin, and GNP will improve biocompatibility, shield the negative surface charge, and prolong circulation half-life. Pegylation of PLGA has been demonstrated to dramatically decrease serum protein adsorbtion (Muller, M et. al. (2003) J. Biomed. Mater. Res. A V66. 55-61.) and reduce uptake by macrophages in the bloodstream (Faraasen, S et. al. (2003) Pharm. Res. V20. 237-246). Similarly, albumin has been shown to prolong circulation half-life of drugs and proteins injected systemically (Dennis, M S et. al. (2002) Journal of Biological Chemistry V277. 35035-35043.; Lu, W et. al. (2005) J. Control Release V107. 428-448.) and as stated previously, can also facilitate transcytotic and endocytotic activity of bound ligands.

In a further embodiment iron oxide particles are blended into the nanosphere core to create magnetizable PLGA nanospheres capable of being delivered to magnetic devices, compounds, and implants.

Methyl and laurel ester end-capped PLGA polymers and co-polymers can be used to further reduce nanovesicle surface charge, by reducing or removing carboxylic acid groups.

Synthetic polymer nanovesicles with encapsulated carrier-NAA compounds will be conjugated with surface ligands to increase delivery of encapsulants to tissues and cells. In preferred embodiments, PLGA nanovesicles with encapsulated carrier-NAA compounds will be conjugated with ligands including TAT-PTD (and derivatives thereof), AAV tropism factor, other cell penetrating and targeting peptides, antibodies, transferrin, ICAM, folic acid, and combinations thereof.

In a further embodiment, synthetic polymer nanovesicles with encapsulated carrier-NAA compounds will be conjugated with ApoE2, which as explained facilitates the transport of compounds by utilizing endocytotic and/or transcytotic mechanisms of transport.

In a further embodiment of the nanovesicle delivery system, synthetic polymer nanovesicles with encapsulated carrier-NAA compounds will be labeled with functionalized PEG-PEI copolymers. Depending on the PEG-PEI formulation, coating of nanovesicles with functionalized PEG-PEI copolymers may improve nanovesicle functionality by reduction of nanovesicle surface charge, improved biocompatibility, and enhanced delivery to tissues and cells.

Pegylation of the nanospheres can be performed using specific formulations of PEG-PEI that exhibit a low positive surface charge by adsorption to the PLGA nanospheres stabilized by ionic interaction. The effectiveness of adsorption is in part dependent of the MW of PEI, PEG, and the PEG:PEI ratio. Copolymer coatings may be comprised of branched or linear PEI (MW range from 100 to 2000 Da) and PEG chains (MW range from about 200 Da to 10 kDa).

Proteins and peptides which contain at least a single free sulfhydryl group or peptides with a single cystein end group can be conjugated directly to PEG-PEI copolymers that are used to coat the nanosphere surfaces using a commercially available NHS-PEON-MAL bifunctional linker (Pierce). The PEO is a spacer arm that can range in MW from about 400-900 Da. The spacer arm in this linker enables more freedom for ligands to bind to their target sites and reduces the chance of sterically blocking the active site of the ligand.

Alternatively, EDC chemistry can be used to couple carboxyl groups of proteins and peptides to amine groups of PEG-PEI polymers which are then used to coat the nanosphere surface.

In a preferred embodiment, PLGA nanovesicles containing encapsulated carrier-NAA compounds will be labeled with derivatives of PEI2K-PEG5K copolymers, wherein the copolymer may contain additional functional groups for improved delivery to tissues and cellular uptake. Example 7 describes the preparation of TAT-PEG-PEI copolymer to PLGA nanovesicles with encapsulated PEI2K(PEG5K)₁₀-NAA polyplex.

Release kinetics of carrier-NAA encapsulants from PLGA nanovesicles can be altered by using or blending different MW PLGA ranging from about 8 to 100 kDa. Different monomer ratios of lactide and glycolide that compose PLGA can be used to alter release rates of compounds. PLGA monomer ratios ranging from (50:50) to (100:0) lactide to glycolide can be used for nanovesicle formulations. Increasing the salt concentration during synthesis can alter the internal osmotic pressure within the nanovesicle that can affect the release of encapsulated compounds.

PLGA nanovesicle size can be modulated by varying the sonication intensity and duration when forming the double emulsion. Longer durations and higher amplitudes create smaller nanospheres particles. Nanosphere size can be altered using different MW and concentrations of PVA. Higher MW and higher PVA concentrations generally provide smaller particles.

Cell Targeting Ligands

In certain embodiments, it is desirable to functionalize polyplexes and nanovesicles using targeting moieties that are specific to a particular cell type, tissue, and the like. Targeting using a variety of moieties (e.g., ligands, receptors, and monoclonal antibodies) has been described. (see, e.g., U.S. Pat. No. 4,603,044).

Examples of targeting moieties include monoclonal antibodies specific to antigens associated with neoplasms, such as prostate cancer specific antigen and MAGE. Tumors can also be diagnosed by detecting gene products resulting from the activation or over-expression of oncogenes, such as ras or c-erbB2. In addition, many tumors express antigens normally expressed by fetal tissue, such as the alphafetoprotein (AFP) and carcinoembryonic antigen (CEA). Sites of viral infection can be diagnosed using various viral antigens such as hepatitis B core and surface antigens (HBVc, HBVs) hepatitis C antigens, Epstein-Barr virus antigens, human immunodeficiency type-1 virus (HIV1) and papilloma virus antigens. Inflammation can be detected using molecules specifically recognized by surface molecules which are expressed at sites of inflammation such as integrins (e.g., VCAM-1), selectin receptors (e.g., ELAM-1) and the like. Cell targeting agents maybe selected from the group consisting of natural or synthetic ligands, antibodies, antibody fragments or other biomolecules suitable for the purpose.

Cell targeting ligands are any ligand specific for a characteristic component of the targeted region. Preferred targeting ligands include proteins such as polyclonal or monoclonal antibodies, antibody fragments, or chimeric antibodies, enzymes, or hormones, or sugars such as mono-, oligo- and poly-saccharides (see, Heath et al., Chem. Phys. Lipids 40:347 (1986)). For example, disialoganglioside GD2 is a tumor antigen that has been identified on neuroectodermal origin tumors, such as neuroblastoma, melanoma, small-cell lung carcinoma, glioma and certain sarcomas (Mujoo et al., 1986). Liposomes containing anti-disialoganglioside GD2 monoclonal antibodies have been used to aid the targeting of the liposomes to cells expressing the tumor antigen (Pagnan et al., 1999). In another non-limiting example, breast and gynecological cancer antigen specific antibodies are described in U.S. Pat. No. 5,939,277, incorporated herein by reference. In a further non-limiting example, prostate cancer specific antibodies are disclosed in U.S. Pat. No. 6,107,090, incorporated herein by reference. Thus, it is contemplated that the antibodies described herein or as would be known to one of ordinary skill in the art may be used to target specific tissues and cell types in combination with the compositions and methods of the present invention. In certain embodiments of the invention, contemplated targeting ligands interact with integrins, proteoglycans, glycoproteins, receptors or transporters. Suitable ligands include any that are specific for cells of the target organ, or for structures of the target organ exposed to the circulation as a result of local pathology, such as tumors.

Non-limiting examples of synthetic polymers which may be used for the construction of nanovesicles are poly(ester)s, poly(urethane)s, poly(alkylcyanoacrylate)s, poly(anhydride)s, poly(ethylenevinyl acetate), poly(lactone)s, poly(styrene)s, poly(amide)s, poly(acrylonitrile)s, poly(acrylate)s, poly(metacrylate)s, poly(orthoester)s, poly(ether-ester)s, poly(tetrafluoroethylene)s, mixtures thereof and copolymers thereof.

In certain embodiments, the poly(ester) is a member selected from the group consisting of poly(lactic acid) (PLA), poly(glycolic acid) (PGA), and poly(lactide-co-glycolide) (PLGA) and block copolymers (e.g., diblock, triblock, multiblock, and star-shaped block) comprising the biodegradable poly(esters) and poly(ethylene glycol) (PEG).

In certain embodiments, the cationic polymer is a member selected from the group consisting of poly(ethyleneimine), poly(propyleneimine), polyamidoamine dendrimer, poly(allylamine) and derivatives thereof.

Examples of biodegradable PEI derivatives useful in the invention can be found in the art (Gosselin et al., Efficient gene transfer using reversibly cross-linked low molecular weight polyethylenimine, Bioconjug Chem. 2001 November-December; 12(6):989-94). PEI derivatives can be prepared by cross-linking 800 Da PEI with dithiobis(succinimidylpropionate) (DSP) and/or dimethyl 3,3′-dithiobispropionimidate 2HCl (DTBP).

An article (Forrest et al., A degradable polyethylenimine derivative with low toxicity for highly efficient gene delivery, Bioconjug Chem. 2003 September-October; 14(5):934-40)) discloses highly branched 14-30 kDa polycations that are biodegradable analogs of 25 KDa PEI produced by addition of amino groups on 800 Da PEI to diacrylates such as, for example, 1,3-butanediol diacrylate of varying spacer length. These or similar derivatives can also be useful in the invention.

Polymer Microbubbles—Ultrasound Enhanced Delivery of Carrier-NAA

Intravascular and local injection of gas-filled microbubbles followed by application of focused ultrasound has been reported to enhance drug delivery to tissues and cells. Microbubble agents are capable of being disrupted under focused ultrasound pressure, resulting in release of contents as well as transient poration of cell membranes and permeabalization of microvasculature (Howard, C M et. al. (2006) J. Cell Physiol V209.413-421.; Kimmel, E (2006) Crit. Rev. Biomed. Eng V34.105-161.; Kost, J et. al. (1989) Proc. Natl. Acad. Sci. U.S.A V86.7663-7666.; Ter, H G (2006) Prog. Biophys. Mol. Biol. V). Previous studies have shown that ultrasound irradiated microbubbles improved plasmid delivery to myofibers in vitro, and following intramuscular delivery (Chen, Y C et. al. (2006) Ultrasound Med. Biol. V32. 131-137.; Liang, H D et. al. (2004) Ultrasound Med. Biol. V30. 1523-1529.; Lu, Q L et. al. (2003) Gene Ther. V10. 396-405.; Wang, X et. al. (2005) Radiology V237. 224-229). Recent studies have also demonstrated that microbubbles, in conjunction with ultrasound, resulted in the appearance of extravagination points up to 500 nm in size within muscle capillaries (Song, J et. al. (2002) J. Am. Coll. Cardiol. V39. 726-731), which is more than adequate for increasing carrier-NAA uptake across the microvasculature. Thus, for example, microbubble ultrasound will significantly increase the level of dystrophin expression in heart, diaphragm, and limb muscles following systemic delivery of carrier-NAA compounds.

Microbubbles are used as contrast agents that by their very purpose (to increase contrast of an ultrasound image) are subjected to insonation, and ultrasound energy has been shown to have a role in increasing polymer degradation rates. Ultrasound has been used to enhance degradation and drug delivery from both biodegradable and non-biodegradable polymeric devices (Kost, J et. al. (1989) Proc. Natl. Acad. Sci. U.S.A V86. 7663-7666.; Ter, H G (2006) Prog. Biophys. Mol. Biol. V). Both cavitation and acoustic streaming have a role. Various parameters, including ultrasound frequency, size of incorporated drug particles, and the porosity of the polymeric matrix have been investigated and related to the ultrasound drug release enhancement effect in a non-biodegradable polymer device. The ability to externally control degradation and drug release rates opens many doors to noninvasive, targeted drug delivery systems. Additionally, ultrasound is a relatively safe triggering mechanism. These systems have great potential for targeted treatment of diseases such as cancer, for which the current systemic treatments have severe toxic side effects.

The invention provides synthetic polymer microbubbles that are loaded with carrier-NAA compounds and NAAs, whereby contents can be released by exposure to ultrasound, further wherein the synthetic polymer nanovesicle is PLGA. Effective encapsulation of PEG-PEI-NAA into microbubbles has been shown. These compounds, which are being developed for US-triggered release make an attractive alternative to the slow release synthetic polymer nanovesicles. These synthetic polymer microbubbles will not only improve circulation time of their cargo, but upon ultrasound insonation the microbubbles will break apart and release their cargo in a spatially and temporally controlled manner. In addition, the insonated microbubbles will cause physical disruption of microvasculature for enhanced delivery to target tissue interstitium and cells.

Functionalized Oligonucleotides

The PEG-PEI copolymers and PLGA compounds described are applicable for the delivery of a variety of NAAs including 2′OMeAOs, phosphorothioate oligonucleotides, siRNA, phosphodiester oligonucleotides, PNAs, and ribozymes. In a further embodiment, the invention provides for the use of novel carrier functionalized double-stranded oligonucleotides (CFOs) comprised of a functionalized sense strand carrier oligonucleotide matched with a complimentary antisense oligonucleotide. In a referred embodiment a CFO is comprised of an antisense strand.

CFOs are double-stranded compounds that consist of an antisense oligonucleotide (AO) annealed by Watson-Crick base pairing to a sense carrier strand (SSCO) that may be functionalized. In one preferred embodiment, the AO is chosen from specific sequences that modulate pre-mRNA or mRNA splicing. The SSCO is chosen to be mostly but not completely complimentary to the AO. Once the CFO is in the nucleus of the target cell, Watson-Crick base pairing between the AO and its fully complimentary site on the target gene will be favored energetically, enabling release of the SSCO from the AO. The use of CFOs has several potential advantages over single-stranded oligonucleotides. First, the double-stranded CFO will have significantly greater stability against nuclease degradation. Second, the AO can be composed of 2′O-methyl, morpholino, or other chemistries. Morpholinos are uncharged synthetic oligonucleotides that are extraordinarily resistant to degradation. Third, the SSCO can be readily conjugated to moieties that can enhance transport across the microvasculature and/or cellular uptake, and/or nuclear localization. For these reasons the CFOs represent a novel alternative approach that may mitigate potential problems associated with oligonucleotide degradation and may greatly improve delivery profile of oligonucleotides.

The invention comprises CFOs where carrier strand that may contain a targeting group which has an effect selected from the group consisting of increasing delivery of the AO to tissues, delivery of the AO across the microvasculature, cellular uptake of the AO, nuclear localization of the AO, and combinations thereof. Non-limiting examples of targeting groups are TAT-PTD (and derivatives thereof), AAV tropism factor, nuclear localization signal (NLS) peptide, cell targeting peptides, and combinations thereof.

Molecular Therapy

Molecular therapy NAAs can be delivered in vivo by administration to an individual patient, typically by systemic administration (e.g., intravenous, intraperitoneal, intramuscular, subdermal, or intracranial infusion) or topical application. Alternatively, NAAs can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, usually after selection for cells which have incorporated the NAA agents.

Ex vivo cell transfection for diagnostics, research, or for molecular therapy (e.g., via re-infusion of the transfected cells into the host organism) is well known to those of skill in the art. In a preferred embodiment, cells are isolated from the subject organism, transfected with a polyplex or nanovesicle, and re-infused back into the subject organism (e.g., patient). Various cell types suitable for ex vivo transfection are well known to those of skill in the art (see, e.g., Freshney et al., Culture of Animal Cells, A Manual of Basic Technique (3rd ed. 1994)) and the references cited therein for a discussion of how to isolate and culture cells from patients).

In one embodiment, stem cells are used in ex vivo procedures for cell transfection and molecular therapy. The advantage to using stem cells is that they can be differentiated into other cell types in vitro, or can be introduced into a mammal (such as the donor of the cells) where they will engraft in the bone marrow. Methods for differentiating CD34+ cells in vitro into clinically important immune cell types using cytokines such a GM-CSF, IFN-.gamma. and TNF-.alpha. are known (see Inaba et al., J. Exp. Med. 176:1693-1702 (1992)).

Stem cells are isolated for transduction and differentiation using known methods. For example, stem cells are isolated from bone marrow cells by panning the bone marrow cells with antibodies which bind unwanted cells, such as CD4+ and CD8+ (T cells), CD45+(panB cells), GR-1 (granulocytes), and lad (differentiated antigen presenting cells) (see Inaba et al., J. Exp. Med. 176:1693-1702 (1992)).

Therapeutic polyplexes and/or nanovesicles comprising NAAs can also be administered directly to an organism for transduction of cells in vivo. Administration is by any of the routes normally used for introducing a molecule into ultimate contact with blood or tissue cells including, but not limited to, injection, infusion, topical application and electroporation. Suitable methods of administering such therapeutic polyplexes and/or nanovesicles comprising NAAs are available and well known to those of skill in the art, and, although more than one route can be used to administer a particular composition, a particular route can often provide a more immediate and more effective reaction than another route. Methods for introduction of DNA into hematopoietic stem cells are disclosed, for example, in U.S. Pat. No. 5,928,638.

Pharmaceutically acceptable carriers are determined in part by the particular composition being administered, as well as by the particular method used to administer the composition. Accordingly, there is a wide variety of suitable formulations of pharmaceutical compositions available, as described below (see, e.g., Remington's Pharmaceutical Sciences, 17th ed., 1989).

Dosages

For therapeutic applications, the dose of polyplexes and/or nanovesicles and NAA administered to a patient, or to a cell which will be introduced into a patient, in the context of the present disclosure, should be sufficient to effect a beneficial therapeutic response in the patient over time. In addition, particular dosage regimens can be useful for determining phenotypic changes in an experimental setting, e.g., in functional genomics studies, and in cell or animal models. The dose will be determined by the efficacy and Kd of the particular polyplexes and/or nanovesicles and NAAs employed, the nuclear volume of the target cell, and the condition of the patient, as well as the body weight or surface area of the patient to be treated. The size of the dose also will be determined by the existence, nature, and extent of any adverse side-effects that accompany the administration of a particular compound or oligonucleotide agents in a particular patient.

The maximum therapeutically effective dosage of polyplexes and/or nanovesicles and NAAs for approximately 99% binding to target sites is calculated to be in the range of less than about 1.5×10⁵ to 1.5×10⁶ copies of the specific polyplexes and/or nanovesicles and NAA molecules per cell. The number of polyplexes and/or nanovesicles and NAAs per cell for this level of binding is calculated as follows, using the volume of a HeLa cell nucleus (approximately 1000 m3 or 10-12 L; Cell Biology, (Altman & Katz, eds. (1976)). As the HeLa nucleus is relatively large, this dosage number is recalculated as needed using the volume of the target cell nucleus. This calculation also does not take into account competition for polyplexes and/or nanovesicles and NAA binding by other sites. This calculation also assumes that essentially all of the polyplexes and/or nanovesicles and NAA is localized to the nucleus. A value of 100×Kd is used to calculate approximately 99% binding of to the target site, and a value of 10×Kd is used to calculate approximately 90% binding of to the target site. For this example, Kd=25 nM.

In determining the effective amount of the polyplexes and/or nanovesicles and NAA to be administered in the treatment or prophylaxis of disease, the physician evaluates circulating plasma levels of the polyplexes and/or nanovesicles and NAA, potential polyplexes and/or nanovesicles and NAA toxicities, progression of the disease, and the production of anti-polyplexes and/or nanovesicles and NAA antibodies. Administration can be accomplished via single or divided doses.

Pharmaceutical Compositions and Administration

Administration of therapeutically effective amounts is by any of the routes normally used for introducing polyplexes and/or nanovesicles and NAAs into ultimate contact with the tissue to be treated. The polyplexes and/or nanovesicles and NAA are administered in any suitable manner, preferably with pharmaceutically acceptable carriers. Suitable methods of administering such modulators are available and well known to those of skill in the art, and, although more than one route can be used to administer a particular composition, a particular route can often provide a more immediate and more effective reaction than another route.

Pharmaceutically acceptable carriers are determined in part by the particular composition being administered, as well as by the particular method used to administer the composition. Accordingly, there is a wide variety of suitable formulations of pharmaceutical compositions that are available (see, e.g., Remington's Pharmaceutical Sciences, 17.sup.th ed. 1985)).

The polyplexes and/or nanovesicles and oligonucleotide agent, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Formulations suitable for parenteral administration, such as, for example, by intravenous, intramuscular, intradermal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The disclosed compositions can be administered, for example, by intravenous infusion, orally, topically, intraperitoneally, intravesically or intrathecally. The formulations of compounds can be presented in unit-dose or multi-dose sealed containers, such as ampules and vials. Injection solutions and suspensions can be prepared from sterile powders, granules, and tablets of the kind previously described.

In certain cases, alteration of a genomic sequence in a pluripotent cell (e.g., a hematopoietic stem cell) is desired. Methods for mobilization, enrichment and culture of hematopoietic stem cells are known in the art. See for example, U.S. Pat. Nos. 5,061,620; 5,681,559; 6,335,195; 6,645,489 and 6,667,064. Treated stem cells can be returned to a patient for treatment of various diseases including, but not limited to, SCID and sickle-cell anemia.

Target Genetic Disorders

The polyplexes and/or nanovesicles and NAAs are useful for diagnosis and treatment of genetic disorders in patients. Non-limiting examples of genetic disorders that can be diagnosed and treated using this method include hereditary diseases such as cystic fibrosis, Tay-Sachs disease, Lesch-Nyhan Syndrome, sickle cell anemia, hemophilia, atherosclerosis, diabetes, and obesity. Such hereditary diseases may include degenerative and non-degenerative neurological diseases such as Alzheimer's disease, Parkinson's disease, amyotrophic lateral sclerosis, Huntington's disease, Wilson's disease, spinal cerebellar ataxia, Friedreich's ataxia and other ataxias, prion diseases including Creutzfeldt-Jakob disease, dentatorubral pallidoluysian atrophy, Fibrodysplasia Ossificans Progressiva, spongiform encephalopathies, myotonic dystrophy, Duchene's muscular dystrophy, spinal muscular atrophy, depression, schizophrenia, and epilepsy. Hereditary diseases may also include metabolic diseases such as, for example, hypoglycemia or phenylketonuria. Cardiovascular diseases and conditions are also included, non-limiting examples of which include atherosclerosis, myocardial infarction, and high blood pressure. The invention can further be used for detection and diagnosis of Lyme disease, tuberculosis, and sexually transmitted diseases.

The polyplexes and/or nanovesicles and NAA is further useful for diagnosis and treatment of disorders of clinical interest. Non-limiting examples of target disorders of clinical interest include asthma, arthritis, psoriasis, excema, allergies, drug resistance, drug toxicity, and cancers such as, but not limited to, human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangi oendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g. acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease.

The polyplexes and/or nanovesicles and NAA is further useful for diagnosis and treatment of patients with autoimmune diseases, including but not limited to, insulin dependent diabetes mellitus, multiple sclerosis, systemic lupus erythematosus, Sjogren's syndrome, scleroderma, polymyositis, chronic active hepatitis, mixed connective tissue disease, primary biliary cirrhosis, pernicious anemia, autoimmune thyroiditis, idiopathic Addison's disease, vitiligo, gluten-sensitive enteropathy, Graves' disease, myasthenia gravis, autoimmune neutropenia, idiopathic thrombocytopenia purpura, rheumatoid arthritis, cirrhosis, pemphigus vulgaris, autoimmune infertility, Goodpasture's disease, bullous pemphigoid, discoid lupus, ulcerative colitis, and dense deposit disease.

It is appreciated that the compositions and methods described herein will be useful in diagnosing and treating diseases of other mammals, for example, farm animals including cattle, horses, sheep, goats, and pigs, household pets including cats and dogs, and plants including agriculturally important plants and garden plants.

The invention will be illustrated in more detail with reference to the following Examples, but it should be understood that the present invention is not deemed to be limited thereto.

### EXAMPLES

### Example 1

### Polyplex Stability Measured by Polyanion Competition Assay

In a useful polyplex delivery system, the electrostatic charge association between the NAA and copolymer must be strong enough to promote cellular uptake, but not too strong as to prohibit release so that NAA can be translocated to target cell nuclei. A polyanion competition assay can assess the relative stability, or association-dissociation dynamics, of the various polyplexes (FIG. 1d; see also FIGS. 5 & 6 in (Glodde, M et. al. (2006) Biomacromolecules. V7(1). 347-356)). Heparin, a linear polysaccharide bearing sulfonate groups, can be used as a model polyanion. Polyplexes are incubated with varying amounts of heparin, electrophoresed on agarose gels, and the intensity of the “free NAA” band (i.e., released NAA) was quantified. Surprisingly, we found PEI2K-based polyplexes to be substantially more stable than several of the PEI25K-based polyplexes. The most stable polyplex, PEI2K(PEG5K)₁₀-NAA, had an IC₅₀ value about 60% higher than the most stable PEI25K-based polyplex. The stability of the PEI2K-based polyplexes is one of their salient features that explain their high transfection capacity in vivo.

### Example 2

### Induction of Dystrophin Expression in Skeletal Muscles of mdx Mice Following Intramuscular Injections of PEG-PEI-AO Polyplexes

PEG-PEI-AO Induction of Dystrophin Expression at 3 Weeks Post-Transfection

We measured AO-mediated dystrophin expression in mdx mice at 3 weeks after intramuscular injection of AO complexed with low and high MW PEG-PEI copolymers (Williams, J H et. al. (2006) Mol. Ther. V14(1). 88-96). For these studies we used the 6-FAM-2′O-methyl antisense oligoribonucleotide (2OMeAO) that has previously been shown to induce skipping of exon 23 in mdx mice (Lu, Q L et. al. (2003) Nat. Med. V9. 1009-1014.; Lu, Q L et. al. (2005) Proc. Natl. Acad. Sci. U.S.A V102. 198-203), the site of a point mutation encoding an early termination signal. In this study, mdx mice (8 wks of age) were anesthetized and TA muscles were injected with 20 μg of AO, complexed with the following copolymers: PEI2K(PEG550)₁₀, PEI25K(PEG5K)₂₅, and PEI25K(PEG5K)₅₀. To facilitate comparison with previous studies (Lu, Q L et. al. (2003) Nat. Med. V9. 1009-1014.; Lu, Q L et. al. (2005) Proc. Natl. Acad. Sci. U.S.A V102. 198-203), a group of mice were also injected with 20 μg of AO complexed with the commercial non-ionic polymer F-127. At 3 weeks after transfection dystrophin expression was measured with immunohistochemistry and western analysis.

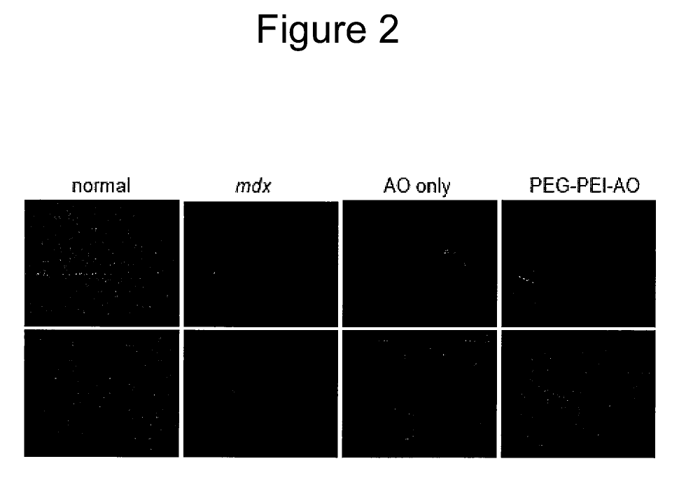

Single injections of 20 μg of AO complexed with PEI2K(PEG550)₁₀ copolymers into TA muscles of mdx mice produced outstanding transfection efficiency as evidenced by the high number dystrophin-positive fibers within muscle transverse sections compared with injections of AO alone (FIG. 2). Dystrophin-positive fibers were broadly, but not uniformly, distributed throughout the muscle cross-section (FIG. 3). In the most heavily transfected regions, nearly 100% of the fibers were dystrophin-positive. In contrast, intramuscular injections of AO complexed with two different high M_{w} PEI25K-based copolymers resulted in significantly fewer dystrophin-positive fibers expression than the polyplexes containing low M_{w} PEI2K(PEG550)₁₀ (FIG. 4). Also, F127-AO injections resulted in nearly 3-fold fewer dystrophin-positive fibers than injections of PEI2K(PEG550)₁₀-AO (FIG. 4).

Western blots revealed that dystrophin expression at 3 wks after injection of PEI2K(PEG550)₁₀-AO was about 5% of age-matched control muscles (FIG. 5, lanes 2-4).

PEG-PEI-AO Induction of Dystrophin Expression Increases Between 3 and 9 Weeks Post-Transfection

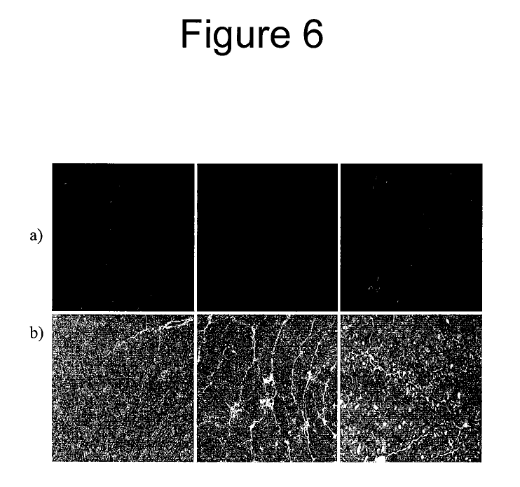

Experiments show that polyplex-mediated induction of dystrophin expression appears to increase in magnitude beyond the 3 wk time point. Transverse muscle sections at 9 wks after injection of PEI2K(PEG550)₁₀-AO show dystrophin-positive fibers distributed throughout the muscle cross-section (FIG. 6). These data suggest that the PEG-PEI copolymers can effectively protect the AO from degradation, and that cellular transfection may be ongoing at 9 wks after IM delivery. Moreover, western analysis of 9 wk muscles (FIG. 5, lanes 9-11) show that dystrophin expression was substantially increased relative to the highest levels obtained with the same polyplex at 3 wks. Average dystrophin expression at 9 wks was 9.7% (N=3), with peak levels reaching 18% of the normal level.

Although PEI alone is known to be cytotoxic to cells, including muscle cells (Bremmer-Bout, M et. al. (2004) Mol. Ther. V10. 232-240), PEGylation of PEI has previously been shown to substantially reduce its cytotoxicity (Petersen, H et. al. (2002) Bioconjug. Chem. V13.845-854.; Shi, L et. al. (2003) Gene Ther. V10. 1179-1188.; Sung, S J et. al. (2003) Biol. Pharm. Bull. V26. 492-500). In support of this view, H&E staining of muscles injected with PEI2K(PEG550)₁₀-AO polyplexes revealed roughly similar, or even lower, levels of proliferative cells compared with mdx control muscles (FIG. 6). Likewise, the H&E sections did not show any extensive areas of regenerating muscle fibers, a hallmark of cytotoxicity. Thus, the high transfection efficiency obtained with low MW PEI2K(PEG550)₁₀-AO polyplexes was accomplished without any indication of toxicity.

### Example 3

### GNP Conjugation to Low MW PEI2K-Based Copolymers Improves Transfection Capacity and Dystrophin Induction by PEG-PEI-Oligonucleotide Polyplexes

GNPs have previously been shown to improve cellular uptake and biocompatibility of polymeric nucleotide carriers (Hainfeld, J F et. al. (2000) J. Histochem. Cytochem. V48. 471-480.; Thomas, M et. al. (2003) Proc. Natl. Acad. Sci. U.S.A V100. 9138-9143.) and internalization of gold nanoparticles into various cell types including muscle cells has been demonstrated (Kaisto, T et. al. (1999) Exp. Cell Res. V253.551-560.; Shukla, R et. al. (2005) Langmuir V21. 10644-10654.; Thomas, M et. al. (2003) Proc. Natl. Acad. Sci. U.S.A V100. 9138-9143). Therefore, we examined the influence of conjugating GNP to low MW PEI2K(PEG550)₁₀ copolymers. Western analysis of muscles 3 wks after transfection with GNP-PEI2K(PEG550)₁₀-AO (FIG. 5, lanes 7-8) showed substantially higher levels of dystrophin expression (mean=15.1%; peak value=20.1%; N=2) than our previous results using unmodified PEI2K(PEG550)₁₀ copolymers. Immunohistochemistry at 9 and 16 wks after IM injections revealed significant numbers of dystrophin-positive fibers (FIG. 7). Moreover, western blots showed that dystrophin expression reached 65% of the level expressed in normal age-matched controls at 9 wks after IM injection (FIG. 8; lane 2).

### Example 4

### Induction of Dystrophin Expression in Skeletal Muscles of mdx Mice Following Intramuscular Injections of High MW PEG-PEI-Oligonucleotide Polyplexes Encapsulated into Degradable PLGA Nanovesicles

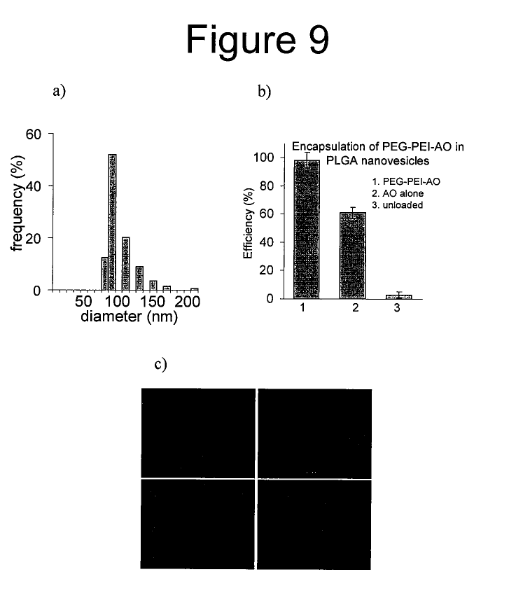

This invention will utilize biodegradable PLGA nanovesicles for delivery of high and low MW PEG-PEI-NAA polyplexes. A detailed explanation of the advantages of this delivery system is provided. Here are data showing extraordinarily high encapsulation efficiency of high MW PEI25K(PEG5K)₁₀-NAA polyplexes into PLGA nanovesicles using a double emulsification (water in oil in water) technique (Cohen-Sacks, H et. al. (2002) Gene Ther. V9. 1607-1616). Parameters of the emulsification procedure were optimized to obtain the smallest possible particles, which should favor their transport across the microvasculature and promote cellular uptake. DLS measurements revealed that the majority of the loaded vesicles were in the size range around 100 nm, although as expected some polydispersity was observed (FIG. 9). The size distribution is consistent with Panyam et al, who demonstrated the encapsulation of plasmid DNA into 100 nm PLGA nanospheres (Panyam, J et. al. (2002) FASEB J. V16.1217-1226). Spectrophotometric measurements revealed nearly 100% encapsulation efficiency (FIG. 9), The high loading efficiency of PEG-PEI-AO is consistent with previous reports of PEI-AO encapsulation in micron sized PLGA vesicles (DeRosa G. et. al. (2003) Biomacromolecules. V4. 529-536). The release kinetics of polyplex from these nanocapsules when measured at 37° C. was about 14 days for 80% release (data not shown), although significantly faster rates of degradation are expected in the body. To our knowledge these data are the first to show successful encapsulation of any cationic polymer-AO polyplexes into nanosized and degradable PLGA vesicles, and as such greatly extend the utility of this drug delivery system.

Experiments show significant numbers of dystrophin positive fibers in TA muscles at 3 wks after a single IM injection of high MW PEI25K(PEG5K)₁₀-AO encapsulated in the PLGA nanovesicles (FIG. 9). Dystrophin expression at such an early time point bodes well for the performance of these “slow release” compounds. These data confirm that encapsulation of the ultra-potent high MW polyplexes in PLGA provides a mechanism for diffusion throughout the muscle volume, followed by sustained release and transfection.

### Example 5

### Dystrophin Expression in Limb Muscles of mdx Mice After Bloodstream Delivery of PEG-PEI-Oligonucleotide Polyplexes

In an experiment an mdx mouse was injected systemically in the tail vein with low MW PEI2K(PEG5K)₁₀-AO polyplex. Western blots from these muscles showed that dystrophin expression reached 20-25% of normal levels (FIG. 8; lanes 3-4). For this experiment mice were given 6 consecutive tail vein injections (1 mg AO each) at 1 wk intervals, and muscles were harvested 2 wks after the final injection. These data document that our polyplexes can induce dystrophin expression after bloodstream delivery. Moreover, the level of dystrophin expression is roughly similar to the expression level reported recently in mdx mice, using 2-fold more morpholino AO than the amount of 2OmeAO used in the present experiment (Alter, J et. al. (2006) Nat. Med. V112. 175-177).

### Example 6

### Double Emulsion Method for Encapsulation of PEI25K(PEG5K)₁₀-NAA Polyplexes into PLGA Nanovesicles

PLGA nanovesicles containing PEG-PEI-NAA polyplex were prepared by an adapted double emulsion (W/O)/W solvent evaporation process discussed in the literature (Cohen-Sack et al). Camphor (7 mg) and PLGA (70 mg) were dissolved in 2 ml of chloroform to form the organic phase. NAA (1 mg of AO) and PEI25K(PEG5K)₁₀ (2.845 mg) were mixed in 300 μl of DI water, bath sonicated for 30 min and incubated on ice for 30 min to form the polyplex in the aqueous phase. The aqueous phase was then added to the organic phase, briefly vortexed, then probe sonicated (using an XL-series, Misonix Inc. sonicator with microtip attachment) at 55 watts for 30 seconds. The single emulsion (W/O) was then poured into a 5% cold PVA solution and probe sonicated again for 1 minute. The double emulsion (W/O/W) was stirred overnight to remove the chloroform. The nanospheres were collected by ultracentrifugation and washed with DI water 3 times. The nanospheres were then resuspended and lyophilized, and stored at −20° C. until used.

### Example 7

### Surface Coating of PLGA Nanovesicles with PEI2K(PEG5K)₁₀ Copolymers and TAT-Functionalized PEI2K(PEG5K)₁₀ Copolymers

PLGA nanospheres (10 mg) with encapsulated PEI25K(PEG5K)₁₀-NAA polyplex (see Example 6) were resuspended in 950 μl of DI water. The solution was briefly bath sonicated to uniformly disperse the nanospheres in solution. PEI2K(PEG5K)₁₀ copolymer (5 mg) was resuspended in 50 μl DI water and added to nanosphere suspension. The suspension was again briefly bath sonicated for 30 seconds and left to incubate on ice for at least 1 hour.

The carboxyl terminus of the HIV-TAT peptide (YGRKKRRQRRR) was covalently coupled to amine groups on PEI2K(PEG5K) 10 copolymer (170 mg) to form TAT-PEI2K(PEG5K)₁₀ copolymer using EDC chemistry. The TAT-PTD (5 mg) was dissolved in 500 μl of 0.1 M MES buffer (pH 5.4). Copolymers (170 mg) were also dissolved in 0.1 M MES buffer. The solutions were mixed together and mixed with 50 μl of EDC (10 mg/ml in water). The solution was incubated at room temperature for 2 hours and the dialyzed for 48 hours at 4° C. in 4 liters of DI water using 10 kDa MW cutoff dialysis tubing. The solution was then lyophilized and stored under nitrogen at −20° C. until used.

### Example 8

### Double Emulsion Method for Polyplex Encapsulated Microcapsule Fabrication

Microcapsules were prepared by an adapted double emulsion (W/O)/W solvent evaporation described previously. Briefly, camphor (0.05 g) and PLGA (0.5 g) were dissolved in 10 mL of methylene chloride, and 1.0 mL of deionized water containing polyplex formed using 1 mg of NAA was added and the polymer solution was probe sonicated at 110 W for 30 s. The resulting (W/O) emulsion was then poured into cold (4° C.), 5% polyvinyl alcohol solution and homogenized (using a PT-3100 Homogenizer, Brinkmann Instruments, with a PTDA3020/2 sawtooth blade) for 5 min at 9500 rpm. The double emulsion (W/O)/W was then poured into a 2% isopropanol solution and stirred at room temperature for 1 h, to evaporate off the methylene chloride, and thus dry the capsules. The capsules were collected by centrifugation, washed one time with deionized water, centrifuged (at 15° C. for 5 min at 5000 g), and the supernatant was discarded. The capsules were then washed three times with hexane to further extract the methylene chloride. The capsules were frozen in an 85° C. freezer and lyophilized, using a Virtis Benchtop freeze dryer. Camphor and water sublime when freeze dried, leaving a void in their place and producing hollow PLGA microcapsules.

While the invention has been described in detail and with reference to specific examples thereof, it will be apparent to one skilled in the art that various changes and modifications can be made therein without departing from the spirit and scope thereof.

What is claimed is:

1. A PEG-PEI-Nucleic Acid Agent (NAA) polyplex comprising a PEG-PEI copolymer, optionally comprising one or more functionalization moieties, and a NAA, wherein the NAA is associated with the copolymer by electrostatic interactions.

2. The PEG-PEI-NAA polyplex of claim 1, wherein the PEI is a branched structure with a Molecular Weight (MW) from about 2 to about 25 kDa.

3. The PEG-PEI-NAA polyplex of claim 1, wherein the MW of PEG ranges from about 500 to about 5000 Da.

4. The PEG-PEI-NAA polyplex of claim 1, wherein the number of PEG chains grafted per molecule of PEI ranges from about 1 to about 25.

5. The PEG-PEI-NAA polyplex of claim 1, wherein the molar ratio of PEI amines (N) to NAA phosphates (P) (N:P ratio) ranges from about 1 to about 15.

6. The PEG-PEI-NAA polyplex of claim 1 comprising one or more functionalization moieties, wherein the one or more functionalization moieties attached to the PEG-PEI copolymer has an effect selected from the group consisting of improving polyplex stability, improving polyplex biodistribution, improving polyplex tissue delivery, improving polyplex cellular uptake, providing cell and/or tissue specificity, and combinations thereof.

7. The PEG-PEI-NAA polyplex of claim 1, wherein the functionalization moiety is a member selected from the group consisting of gold nanoparticles (GNP), TAT-PTD and derivatives thereof, ApoE, albumin, antibody, antibody fragment, magnetic nanoparticle, iron oxide, transferrin, AAV tropism fragment, and combinations thereof.

8. The PEG-PEI-NAA polyplex of claim 1, wherein the NAA is selected from the group consisting of anti sense oligoribonucleotide (AO), oligodeoxynucleotide (ODN), U7-snRNA, siRNA, shRNA, PNA, ribozyme, aptamer, nucleoside 5′ triphosphates, and combinations thereof.

9. The PEG-PEI-NAA polyplex of claim 1, wherein the NAA is an antisense oligoribonucleotide (AO), wherein one or more of the bases is chemically modified, and further wherein the chemical modification is a member selected from the group consisting of 2′O-methyl, phosphorthioate, 2′MEO, phosphodiester, and combinations thereof.

10. The PEG-PEI-NAA polyplex of claim 1, wherein the NAA comprises a carrier-functionalized oligonucleotide (CFO) which comprises an AO hybridized to a partially complimentary carrier strand by Watson-Crick base pairing.

11. The PEG-PEI-NAA polyplex of claim 10, wherein the NAA is a CFO and the carrier strand contains a targeting group which has an effect selected from the group consisting of increasing delivery of the AO to tissues, delivery of the AO across the microvasculature, cellular uptake of the AO, nuclear localization of the AO, and combinations thereof.

12. The PEG-PEI-NAA polyplex of claim 11, wherein the NAA is a CFO and the carrier strand contains a targeting group which is a member selected from the group of TAT-PTD (and derivatives thereof), AAV tropism factor, NLS peptide, cell targeting peptide, and combinations thereof.

13. The PEG-PEI-NAA polyplex of claim 1, wherein PEI has a MW of about 2 kDa; PEG has a MW of about 550 Da; PEG:PEI molar ratio is about 10; and the N:P ratio is about 1 to about 5.

14. The PEG-PEI-NAA polyplex of claim 1, wherein PEI has a MW of about 2 kDa; PEG has a MW of about 5 kDa; PEG:PEI molar ratio is about 10; and the N:P ratio is about 1 to about 5.

15. The PEG-PEI-NAA polyplex of claim 1, wherein PEI has a MW of about 25 kDa; PEG has a MW of about 5 kDa; PEG:PEI molar ratio is about 10; and the N:P ratio is about 2 to about 25.

16. The PEG-PEI-NAA polyplex of claim 1, wherein the functionalization moiety is a member selected from the group consisting of ligands, receptors, monoclonal antibodies, polyclonal antibodies, small molecule ligands, aptamers, and combinations thereof.

17. The PEG-PEI-NAA polyplex of claim 1, wherein the functionalization moiety binds to a protein which is a member selected from the group consisting of tumor-markers, integrins, cell surface receptors, transmembrane proteins, ion channels, membrane transport protein, enzymes, antibodies, and chimeric proteins.

18. The PEG-PEI-NAA polyplex of claim 1, wherein the NAA contains a 2′O-methyl or morpholino AO of sequence 5′-AUUCACUUUCAUAAUGCUGG-3′ (SEQ ID NO: 1) for specific inclusion of human exon 7 of the SMN2 gene.

19. The PEG-PEI-NAA polyplex of claim 1, wherein the NAA contains a 2′O-methyl or morpholino AO of sequence 5′-UCAAGGAAGAUGGCAUUUCU-3′ (SEQ ID NO: 2) for specific skipping of human exon 51 of the dystrophin gene.

20. A synthetic polymer nanovesicle encapsulating either PEG-PEI-NAA polyplex or NAA alone, wherein the nanovesicle optionally comprises surface modifications and attached moieties for delivery of NAA to tissues and cells.

21. The synthetic polymer nanovesicle of claim 20, wherein the synthetic polymer is a member selected from the group consisting of poly(lactic acid) (PLA), poly(glycolic acid) (PGA), and poly(lactic-co-glycolic acid) (PLGA).

22. The synthetic polymer nanovesicle of claim 20, wherein the encapsulant is a PEG-PEI-NAA polyplex.

23. The synthetic polymer nanovesicle of claim 20, wherein the NAA is a member selected from the group consisting of antisense oligoribonucleotide (AO), oligodeoxynucleotide (ODN), U7-snRNA, siRNA, shRNA, PNA, ribozyme, aptamer, nucleoside 5′ triphosphates, and combinations thereof.

24. The synthetic polymer nanovesicle of claim 20, wherein the NAA is an antisense oligoribonucleotide (AO), and wherein one or more of the bases is chemically modified, and further wherein the chemical modification is a member selected from the group consisting of 2′O-methyl, phosphorthioate, 2′MEO, phosphodiester, and combinations thereof.

25. The synthetic polymer nanovesicle of claim 20, wherein the NAA is a CFO.

26. The synthetic polymer nanovesicle of claim 20, wherein the surface properties of the synthetic polymer nanovesicle is modified by attachment of a compound selected from the group consisting of PEG, GNP, ApoE, transferrin, albumin, and combinations thereof.

27. The synthetic polymer nanovesicle of claim 20, wherein the synthetic polymer nanovesicle is modified by attachment of a compound selected from the group consisting of magnetic nanoparticles, iron oxide, and combinations thereof.

28. The synthetic polymer nanovesicle of claim 20, wherein the surface properties of the synthetic polymer nanovesicles are modified by attachment of compounds selected from the group consisting of TAT-PTD and derivatives thereof, AAV tropism factors, antibodies, antibody fragments, and combinations thereof.

29. The synthetic polymer nanovesicle of claim 20, wherein the synthetic polymer nanovesicle is modified by attachment of functionalized PEG-PEI copolymers.

30. The synthetic polymer nanovesicle of claim 29, wherein the PEG-PEI copolymer comprises PEI with a MW of about 100 to about 2000 Da, PEG with a MW of about 200 to about 10000 Da, and the PEG:PEI ratio is about 1:25.

31. The synthetic polymer nanovesicle of claim 30 wherein the PEG-PEI copolymer comprises a functionalization moiety selected from GNP, TAT-PTD and derivatives thereof, AAV tropism factor, NLS peptide, cell targeting peptide, cell penetrating peptides, and combinations thereof.

32. The synthetic polymer nanovesicle of claim 20 wherein the mean diameter of the synthetic polymer nanovesicle is about 80 to about 200 nm.

33. The synthetic polymer nanovesicle of claim 20, wherein the functionalization moiety of the nanovesicle comprises a protein selected from the group consisting of tumor-markers, integrins, cell surface receptors, transmembrane proteins, ion channels, membrane transport protein, enzymes, antibodies, chimeric proteins, and combinations thereof.

34. A method of making a synthetic polymer nanovesicle comprising a synthetic polymer with encapsulated PEG-PEI-NAA polyplex or NAA alone, wherein the synthetic polymer is functionalized with surface coatings and moieties comprising:
providing a synthetic polymer nanovesicle wherein the synthetic polymer is functionalized with surface coatings and moieties;
providing PEG-PEI-NAA polyplex or NAA alone;
encapsulating the PEG-PEI-NAA polyplex or NAA alone in the synthetic polymer nanovesicle.

35. The method of claim 34, wherein the nanovesicle is biologically degradable, chemically degradable, or both biologically and chemically degradable.

36. The PEG-PEI-Nucleic Acid Agent (NAA) polyplex of claim 1, further comprising a synthetic polymer nanovesicle in the form of a microbubble encapsulating the PEG-PEI-NAA polyplex, wherein release of encapsulant from the microbubble is triggered by ultrasound.

37. The polyplex of claim 36, wherein the synthetic polymer is a member selected from the group consisting of poly(lactic acid) (PLA), poly(glycolic acid) (PGA), and poly(lactic-co-glycolic acid) (PLGA).


SEQUENCE LISTING (2 sequences)

SEQ ID NO: 1 - RNA, 20 nt, Homo sapiens
auucacuuucauaaugcugg

SEQ ID NO: 2 - RNA, 20 nt, Homo sapiens
ucaaggaagauggcauuucu